(19) United States
(12) Patent Application Publication
(10) Pub. No.: US 2024/0378735 A1
(43) Pub. Date: Nov. 14, 2024

(54) REGISTRATION OF IMAGES ACQUIRED WITH DIFFERENT ENDOSCOPIC UNITS

(71) Applicant: SurgVision GmbH, Munich (DE)

(72) Inventors: Adrian TARUTTIS, Munich (DE); Maximilian KOCH, Munich (DE); Dmitry BOZHKO, Munich (DE); Gergana KRATUNCHEVA, Munich (DE)

(21) Appl. No.: 18/692,258

(22) PCT Filed: Sep. 12, 2022
(86) PCT No.: PCT/EP2022/075238

§ 371 Date: Mar. 14, 2024

(30) Foreign Application Priority Data
Sep. 16, 2021 (EP) 21197266.6

(51) Int. Cl. G06T 7/38; A61B 1/00; A61B 1/04; G06T 7/20; G06V 10/774; G06V 10/82

(52) U.S. Cl. CPC G06T 7/38; A61B 1/000096; A61B 1/043; G06T 7/20; G06V 10/774; G06V 10/82; G06T 2207/10016; G06T 2207/10024; G06T 2207/10068; G06T 2207/20081; G06T 2207/20084; G06T 2207/30096

(57) ABSTRACT

A solution is proposed for imaging a body-part (103) of a patient (106) with an endoscopic system (100). A corresponding method (500) comprises acquiring (509-515) two sequences of images with different probes (127n,127b), of corresponding endoscopic units (115m,115b), that are movable therebetween. Corresponding images of each pair in the two sequences are registered (518-584), for example, according to corresponding motions of their probes (127n,127b) that are estimated independently each according to the corresponding images. A method (800) is also proposed for training a neural network (439) that may be used to register the images. Corresponding computer programs (400;700) and computer program products for operating the endoscopic system (100) and for training the neural network (439) are proposed. Moreover, corresponding endoscopic system (100) for imaging the body-part (103), endoscopic equipment (115b) comprising one of the endoscopic units (115m,115b), computing device (242b) for operating the endoscopic system (100) and computing system (600) for training the neural network (439) are proposed. A surgical method, a diagnostic method and a therapeutic method based on the same solution are further proposed.

### TECHNICAL FIELD

The present disclosure relates to the field of medical equipment. More specifically, this disclosure relates to endoscopes.

### BACKGROUND ART

The background of the present disclosure is hereinafter introduced with the discussion of techniques relating to its context. However, even when this discussion refers to documents, acts, artifacts and the like, it does not suggest or represent that the discussed techniques are part of the prior art or are common general knowledge in the field relevant to the present disclosure.

Endoscopes are commonly used in several medical procedures (endoscopies) for imaging body-parts of patients that are not visible normally, for example, for diagnostic, surgical or therapeutic purposes (such as in gastroscopy to investigate symptoms affecting a digestive tract and possibly resecting tumors).

For this purpose, any endoscope comprises a (typically flexible) elongated probe that is inserted directly into a cavity defined by a body-part of a patient (through a natural orifice or a small incision). A tip of the probe is used to reach a region of interest of the body-part for the corresponding medical procedure (a portion of the digestive tract with a possible tumor in the example at issue). The tip of the probe allows illuminating the region of interest and acquiring images thereof, which images are then provided to a monitor for their display. The probe also has one or more working channels, through which different tools may be inserted for use during the medical procedure (for example, for taking samples of the (suspected) tumor or resecting the (identified) tumor). The endoscope allows a physician to look inside the cavity during the medical procedure, at the same time with minimal trauma for the patient.

Recently, endoscopic techniques based on fluorescence imaging have been introduced in clinical researches (for example, for improving early detection of tumors). Fluorescence imaging exploits a fluorescence phenomenon, which occurs in fluorescence substances (called fluorophores) emitting fluorescence light when they are illuminated. In this case, a fluorescence agent (adapted to reaching a specific target, the tumor in this case, and then remaining immobilized thereon) is administered to the patient. Images of the region of interest of the body-part are then acquired and provided to a monitor for their display; in this case, the images are defined by the fluorescence light that is emitted from the region of interest of the body-part, and then represent the fluorophores that are present therein. Therefore, the representation of the (immobilized) fluorescence agent in these images facilitates the identification (and quantification) of the target.

It is also possible to exploit the above-mentioned endoscopic techniques, differentiated as standard (endoscopic) technique and fluorescence (endoscopic) technique, respectively, together. Particularly, an endoscope based on the standard technique may be used to acquire and display (reflectance) images on its monitor and an endoscope based on the fluorescence technique may be used to acquire and display (fluorescence) images on its monitor.

Moreover, a simplified endoscopic unit based on the fluorescence technique, referred to as babyscope, may be inserted into a working channel of the endoscope based on the standard technique, referred to as motherscope. This is cost effective, since it allows using the motherscope already available with the addition of the (simpler) babyscope that exploits the other functionalities of the motherscope (such as for its steering within the cavity). Additionally, this allows the combination of a chip-on-tip imaging technique utilized in the motherscope (video-scope) with an imaging through a fiber bundle utilized in the babyscope (providing enhanced sensitivity). Moreover, the same motherscope (already inserted into the cavity of the patient) may be used for other different medical procedures. For example, it is possible to start inserting the babyscope into the working channel of the motherscope for analyzing a possible tumor. Later on, the babyscope is removed and another tool is inserted into the same working channel, or the babyscope is maintained within the motherscope and the other tool is inserted into an additional working channel, said further tool being used, for example, to take samples of the (suspected) tumor or to resect the (identified) tumor. In this case as well, the reflectance images acquired by the motherscope are displayed on its monitor and the fluorescence images acquired by the babyscope are displayed on its monitor.

However, the correlation of the information provided by the reflectance images and by the fluorescence images displayed on the different monitors may be challenging. This is further hindered by the fact that the reflectance images and the fluorescence images significantly differ, for example, in terms of shape, zooming, resolution, contrast, noise and so on. Moreover, the babyscope is generally not fixed within the working channel of the motherscope; therefore, unknown movements (for example, reciprocal rotations and translations) between the babyscope and the motherscope occur during the medical procedure, for example, due to peristalsis and unavoidable motions of the patient. As a result, corresponding misalignments are generated between the luminescence images and the reflectance images that are misleading for the physician.

### SUMMARY

A simplified summary of the present disclosure is herein presented in order to provide a basic understanding thereof; however, the sole purpose of this summary is to introduce some concepts of the disclosure in a simplified form as a prelude to its following more detailed description, and it is not to be interpreted as an identification of its key elements nor as a delineation of its scope.

In general terms, the present disclosure is based on the idea of registering images acquired with different endoscopic units.

Particularly, an aspect provides a method for imaging a body-part of a patient with an endoscopic system. The method comprises acquiring two sequences of images with different probes (of corresponding endoscopic units) that are movable therebetween. Corresponding images of each pair in the two sequences are registered, for example, according to corresponding motions of their probes that are estimated independently each according to the corresponding images.

A further aspect provides a method for training a neural network that may be used to register the images.

A further aspect provides a computer program for operating the endoscopic system.

A further aspect provides a corresponding computer program product.

A further aspect provides a computer program for training the neural network.

A further aspect provides a corresponding computer program product.

A further aspect provides a corresponding endoscopic system for imaging the body-part.

A further aspect provides an endoscopic equipment comprising one of the endoscopic units.

A further aspect provides a computing device for operating the endoscopic system.

A further aspect provides a computing system for training the neural network.

A further aspect provides a corresponding surgical method.

A further aspect provides a corresponding diagnostic method.

A further aspect provides a corresponding therapeutic method.

More specifically, one or more aspects of the present disclosure are set out in the independent claims and advantageous features thereof are set out in the dependent claims, with the wording of all the claims that is herein incorporated verbatim by reference (with any advantageous feature provided with reference to any specific aspect that applies mutatis mutandis to every other aspect).

### BRIEF DESCRIPTION OF THE DRAWINGS

The solution of the present disclosure, as well as further features and the advantages thereof, will be best understood with reference to the following detailed description thereof, given purely by way of a non-restrictive indication, to be read in conjunction with the accompanying drawings (wherein, for the sake of simplicity, corresponding elements are denoted with equal or similar references and their explanation is not repeated, and the name of each entity is generally used to denote both its type and its attributes, such as value, content and representation). Particularly:

FIG. 1 shows a pictorial representation of an endoscopic system according to an embodiment of the present disclosure;

FIG. 2 shows a functional block diagram of the endoscopic system that may be used to practice the solution according to an embodiment of the present disclosure;

FIG. 3 shows the general principles of the solution according to an embodiment of the present disclosure,

FIG. 4 shows the main software components that may be used to implement the solution according to an embodiment of the present disclosure,

FIG. 5 shows an activity diagram describing the flow of activities relating to an implementation of the solution according to an embodiment of the present disclosure,

FIG. 6 shows a schematic block diagram of a computing system that may be used to train a neural network of the solution according to an embodiment of the present disclosure,

FIG. 7 shows the main software components that may be used to train the neural network in the solution according to an embodiment of the present disclosure,

FIG. 8 shows an activity diagram describing the flow of activities relating to the training of the neural network in the solution according to an embodiment of the present disclosure, and

FIG. 9A-FIG. 9B show different examples of application of the solution according to an embodiment of the present disclosure.

### DETAILED DESCRIPTION

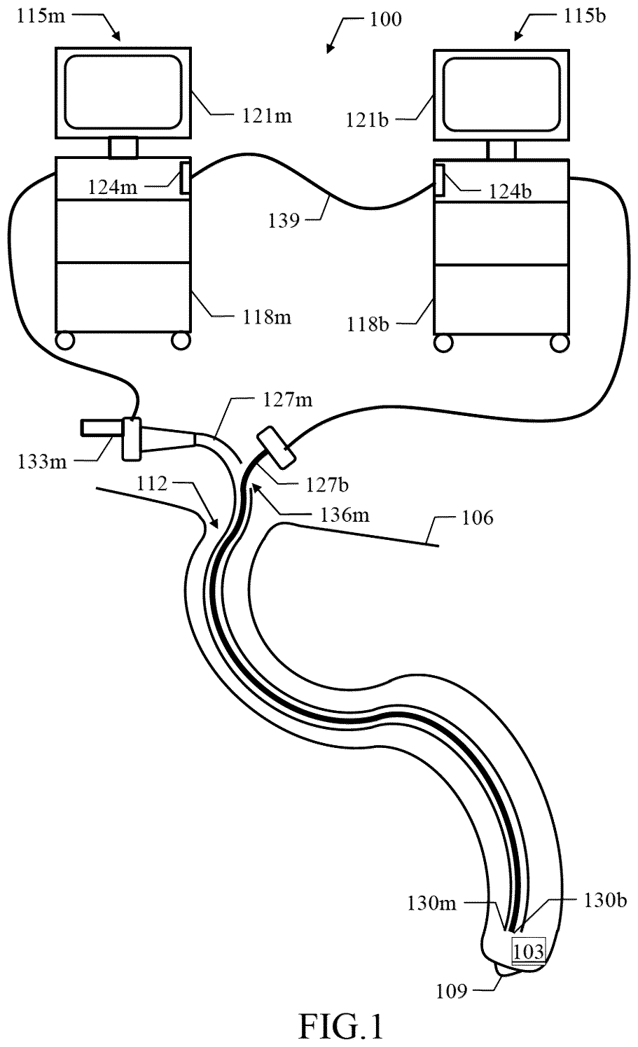

With reference in particular to FIG. 1, a pictorial representation is shown of an endoscopic system 100 according to an embodiment of the present disclosure.

The endoscopic system, or simply endoscope, 100 is used in a medical procedure for imaging a body-part 103 of a patient 106 in a cavity delimited by it (which cavity of the body-part 103 is not visible normally). The body-part 103 comprises a target of a medical procedure, for example, a lesion, such as a tumor 109 to be inspected, resected or treated; the cavity of the body-part 103 is accessible through an opening 112, being either a natural orifice of the patient 106 or a small incision in the skin thereof. For example, in diagnostic applications the endoscope 100 allows discovering/monitoring lesions, in (minimally invasive) surgical applications the endoscope 100 allows identifying lesions to be resected and in therapeutic applications the endoscope 100 allows delineating lesions to be treated. Examples of these medical procedures are gastroscopy, colonoscopy, esophagoscopy and so on for the diagnostic applications, they are arthroscopy, laparoscopy, thoracoscopy and so on for the surgical applications, and they are cauterization, dilatation, stenting and so on for the therapeutic applications.

The endoscope 100 has an unconventional structure that is used to apply a fluorescence (endoscopic) technique (for displaying a fluorescence substance, for example, a fluorescence agent adapted to accumulating in tumors that has been previously administered to the patient 106) in combination with a standard (endoscopic) technique (for displaying what is visible to human eye). For this purpose, the endoscope 100 is composed of two endoscopic units, i.e., a (first) main endoscopic unit 115m (which alone is a conventional device used in clinical practice) combined with a (second) auxiliary endoscopic unit 115b.

The main endoscopic unit, or simply motherscope, 115m comprises the following components. A central unit of the motherscope 115m is used to manage its operation. For example, the central unit is implemented as a trolley 118m, with four casters arranged at corresponding lower corners thereof to facilitate moving it (with a foot brake, not visible in the figure, that is provided for securing the trolley 118m in position). A monitor 121m (for example, mounted on top of the trolley 118m) is used to display images of the body-part 103 during the medical procedure. A video interface 124m (for example, a Serial Digital Interface (SDI) port on the back of the trolley 118m) is used to exchange video information with the outside. A probe 127m (coupled with the trolly 118m, for example, via a cable) is used to act on the patient 106. For example, the probe 127m is implemented as an elongated shaft for insertion into the cavity of the body-part 103; the shaft of the probe 127m may be rigid or preferably flexible to allow its sliding through the cavity of the body-part 103 even when the latter has a curved path. A distal end, or tip, 130m of the probe 127m is used to reach a region of interest of the medical procedure within the cavity of the body-part 103 for illuminating it and acquiring (reflectance) images thereof (as described in the following). A proximal end of the probe 127m (outside the cavity 103) is provided with a handle 133m for driving the tip 130m (via control cables, not shown in the figure). The probe 127m has one or more working channels (accessible through corresponding one or more working ports close to its proximal end), only one shown in the figure wherein it is denoted with the reference 136m. The working channels allow inserting different tools to be used during the medical procedure (for example, snares, forceps, knife, clip applier and so on); a dedicated working channel may also be connected to a fluid injector/extractor (not shown in the figure) for cleaning the tip 130m and the cavity of the body-part 103 during the medical procedure.

The auxiliary endoscopic unit, or simply babyscope, 115b comprises the following components. As above, a central unit of the babyscope 115b is used to manage its operation. For example, the central unit is implemented as a trolley 118b (with four casters to facilitate moving it and a foot brake for securing the trolley 118b in position). A monitor 121b (for example, mounted on top of the trolley 118b) is used to display (further) images of the cavity of the body-part 103 during the medical procedure. A video interface 124b (for example, an SDI port on the back of the trolley 118m) is used to exchange video information with the outside. A probe 127b (coupled with the trolley 118b, for example, via a cable) is used to act on the patient 106. For example, the probe is implemented as a (preferably flexible) elongated shaft. A distal end, or tip, 130b of the probe 127b is used to reach the same region of interest of the medical procedure within the cavity of the body-part 103 for illuminating it and acquiring (fluorescence and possibly reflectance) images thereof (as described in the following).

In a specific implementation of the solution according to an embodiment of the present disclosure, the probe 127b is thinner than the probe 127m. The probe 127b is inserted into the working channel 136m, until its tip 130b reaches the tip 130m of the probe 127m (without impairing a maneuverability of the latter thanks to its size and flexibility). Moreover, the trolley 118m and the trolley 118b are coupled to each other, for example, via a cable 139, to exchange information during the medical procedure.

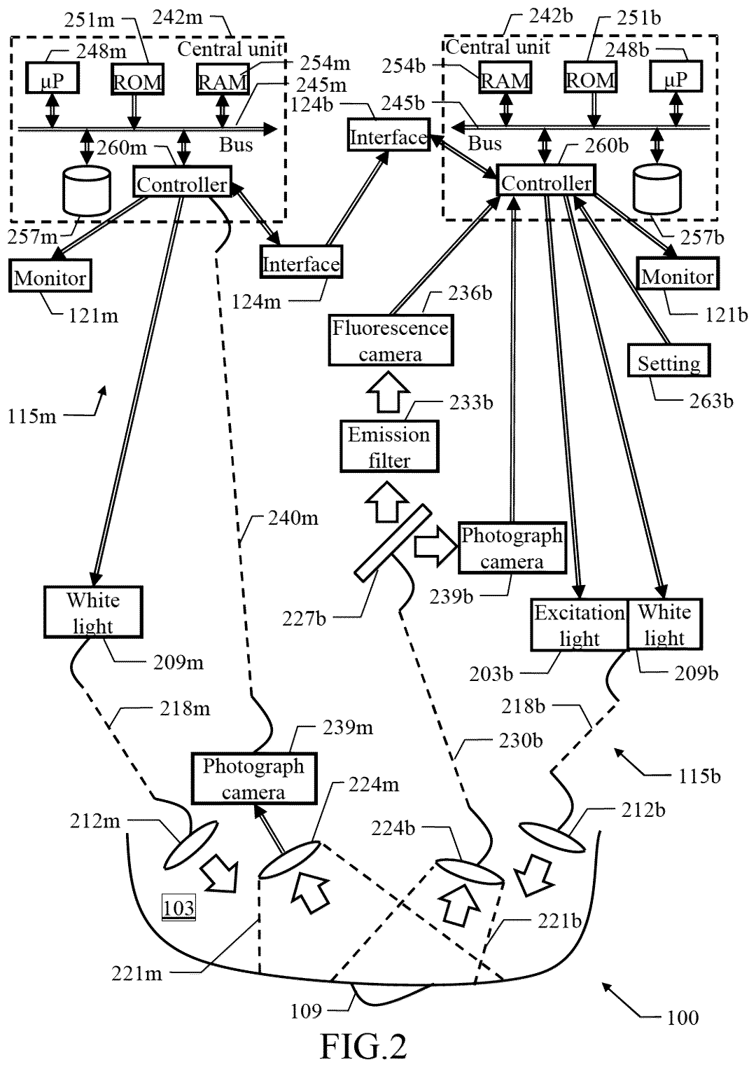

With reference now to FIG. 2, a functional block diagram is shown of the endoscope 100 that may be used to practice the solution according to an embodiment of the present disclosure.

Starting from the babyscope 115b, it comprises the following components. An illumination unit is used to illuminate the region of interest of the cavity 103. For this purpose, an excitation light source 203b (inside the trolley of the babyscope 115b, not shown in the figure), for example, laser-based, generates an excitation light of fluorescence substances; particularly, the excitation light has wavelength and energy suitable to excite the fluorophores of the fluorescence agent (such as of Near Infra-Red (NIR) type). Optionally, a white light source 209b (inside the trolley), for example, of Xenon type, generates a white light (appearing substantially colorless to the human eye, such as containing all the wavelengths of the spectrum that is visible to the human eye at equal intensity), which white light is mixed with the excitation light. Delivery optics 212b (at the tip of the probe of the babyscope 115b, not shown in the figure) delivers the excitation light and the possible white light to the region of interest of the body-part 103. An incoherent bundle of optical fibers 218b (along the probe) transmits the excitation light from the excitation light source 203b, possibly mixed with the white light from the white light source 209b, to the delivery optics 212b (alternatively, not shown in the figure, two separate pieces of delivery optics with corresponding incoherent bundles of optical fibers are used to deliver the excitation light and the white light independently). An acquisition unit is used to acquire the (fluorescence and possibly reflectance) images of the region of interest of the body-part 103 within a field of view thereof 221b (i.e., a part of the world within a solid angle to which the acquisition unit is sensitive), comprising the target 109 in the example at issue. For this purpose, collection optics 224b collects light from the field of view 221b (in an epi-illumination geometry). The collected light comprises fluorescence light that is emitted by any fluorophores present in the field of view 221b (illuminated by the excitation light). Indeed, the fluorophores pass to an excited (electronic) state when they absorb the excitation light; the excited state is unstable, so that the fluorophores very shortly decay therefrom to a ground (electronic) state, thereby emitting the fluorescence light (at a characteristic wavelength, longer than the one of the excitation light because of energy dissipated as heat in the excited state) with an intensity mainly depending on the amount of the fluorophores that are illuminated. Moreover, the collected light comprises visible light (in the visible spectrum) that is reflected by any object present in the field of view 221b (illuminated by the white light). A beam-splitter 227b splits the collected light into two channels. For example, the beam-splitter 227b is a dichroic mirror transmitting and reflecting the collected light at wavelengths above and below, respectively, a threshold wavelength between a spectrum of the visible light and a spectrum of the fluorescence light (or vice-versa). A coherent bundle of optical fibers 230b (along the probe) transmits the collected light from the collection optics 224b to the beam-splitter 227b. In the (transmitted) channel of the beam-splitter 227b with the fluorescence light defined by the portion of the collected light in its spectrum, an emission filter 233b filters the fluorescence light to remove any residual component thereof outside the spectrum of the fluorescence light. A fluorescence camera 236b (for example, of EMCCD type) receives the fluorescence light from the emission filter 233b and generates a corresponding fluorescence (digital) image representing the distribution of the fluorophores in the field of view 221b. Optionally, in the other (reflected) channel of the beam-splitter 227b with the visible light defined by the portion of the collected light in its spectrum, a reflectance, or photograph, camera 239b (for example, of CCD type) receives the visible light and generates a corresponding reflectance (digital) image representing what is visible to human eye in the field of view 221b.

Moving to the motherscope 115m, it comprises the following components. As above, an illumination unit is used to illuminate the region of interest of the body-part 103. For this purpose, a white light source 209m (inside the trolley of the motherscope 115b, not shown in the figure), for example, of Xenon type, generates white light. Delivery optics 212m (at the tip of the probe of the motherscope 115m, not shown in the figure) delivers the white light to the region of interest of the body-part 103. An incoherent bundle of optical fibers 218m (along the probe) transmits the white light from the white light source 209m to the delivery optics 212m. An acquisition unit is used to acquire the (reflectance) images of the region of interest of the body-part 103 within a field of view thereof 221m, comprising the target 109 in the example at issue. For this purpose, collection optics 224m collects visible light (in the visible spectrum) that is reflected by any object present in the field of view 221m illuminated by the white light (in an epi-illumination geometry). A reflectance, or photograph, camera 239m (for example, of CCD type) receives the visible light and generates a corresponding reflectance (digital) image representing what is visible to human eye in the field of view 221m. In a video-scope configuration, the reflectance camera 239m is arranged at the tip of the probe, in this case, a digital connection 240m transmits the reflectance image to the trolley (alternatively, not shown in the figure, the reflectance camera is arranged inside the trolley and a coherent bundle of optical fibers transmits the visible light from the collection optics to the reflectance camera).

A central unit 242b and a central unit 242m are used to control operation of the babyscope 115b and of the motherscope 115m, respectively. The central unit 242b,242m comprises several units that are connected among them through a bus structure 245b,245m. Particularly, a microprocessor (μP) 248b,248m, or more, provides a logic capability of the central unit 242b,242m. A non-volatile memory (ROM) 251b,251m stores basic code for a bootstrap of the central unit 2426,242m and a volatile memory (RAM) 254b,254m is used as a working memory by the microprocessor 248b,248m. The central unit 242b,242m is provided with a mass-memory 257b,257m for storing programs and data, for example, a Solid-State-Disk (SSD). Moreover, the central unit 242b,242m comprises a number of controllers 260b,260m for peripherals, or Input/Output (I/O), units. Particularly, the controllers 260b of the babyscope 115b control the excitation light source 203b, the fluorescence camera 236b, the (possible) white light source 209b, the (possible) reflectance camera 239b, the monitor 121b and the video interface 124b; moreover, the controllers 260b may also control a setting device 263b that is used to set a correction to be used for registering the images of the babyscope 115b and the images of the motherscope 115m manually, for example, comprising rotational/linear dials mounted on the probe or the trolley of the babyscope 115b, foot-operated rotational/linear dials or foot padels, all of them operating either in continuous or toggle mode, and so on. The controllers 260m of the motherscope 115 instead control the white light source 209m, the reflectance camera 239m, the monitor 121m and the video interface 124m. In both cases, the controllers 260b,260m may control further peripherals, not shown in the figure, such as a keyboard, a trackball, a drive for reading/writing removable storage units (such as USB keys) and a network interface card (NIC) for connecting to a (communication) network (such as a Local Area Network (LAN)).

Generally, the field of view 221b of the babyscope 115b and the field of view 221m of the motherscope 115m are different. For example, the field of view 221b is smaller than the field of view 221m, with the fields of view 221b and the field of view 221m that overlap, at least in part (both of them comprising the target 109 in the example shown in the figure). In any case, the images of the babyscope 115b and the images of the motherscope 115m generally have different characteristics, for example, in terms of shape, zooming, resolution, contrast, noise and so on; these different characteristics are far more evident between the fluorescence images of the babyscope 115b and the reflectance images of the motherscope 115m because of their different nature. Moreover, the probe of the babyscope 115b is generally not fixed within the working channel of the motherscope 115n (not shown in the figure), being movable with respect thereto (along its longitudinal axis and especially around it). Unavoidable (unknown a priori) movements of the acquisition unit of the babyscope 115b with respect to the acquisition unit of the motherscope 115m thus normally occur, for example, due to peristalsis, breathing and heartbeat of the patient (in combination with the flexible nature of the probes of the motherscope 115m and of the babyscope 115b). As a consequence, corresponding misalignments are generated between the images of the babyscope 115b and the images of the motherscope 115m (for example, translations and especially rotations).

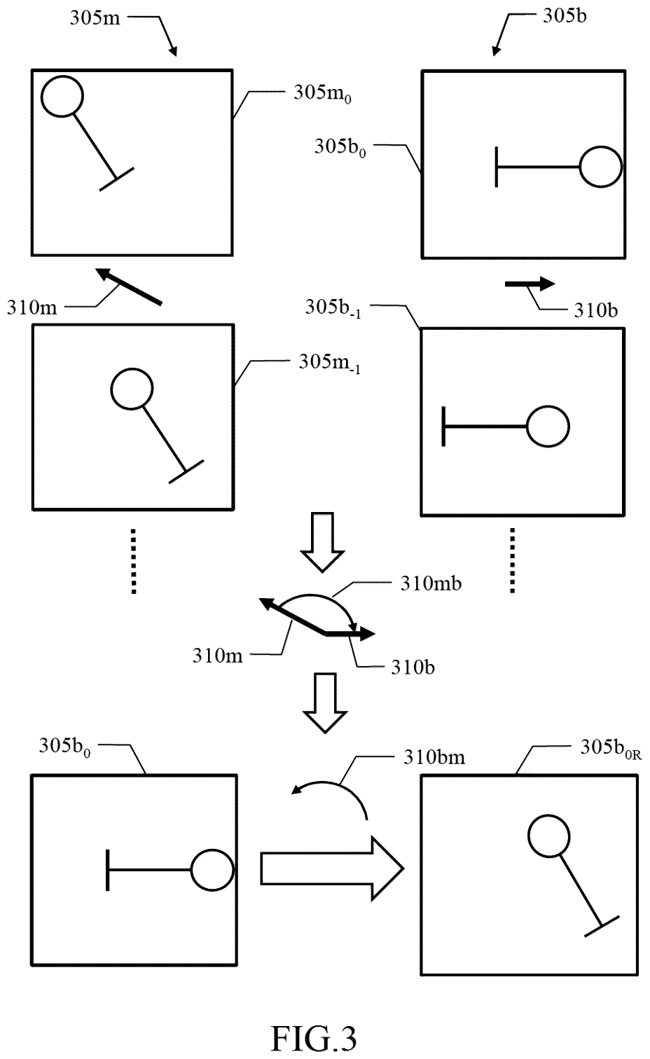

With reference now to FIG. 3, the general principles are shown of the solution according to an embodiment of the present disclosure.

Particularly, the motherscope provides a sequence of reflectance images (motherscope images) 305m that have been acquired over time, only the last two denoted with 305m₀ and 305m₋₁ being shown in the figure. Likewise, the babyscope provides a sequence of corresponding fluorescence and possibly reflectance images (babyscope images) 305b; for the sake of simplicity, the case with only fluorescence images is taken into account, with the figure showing the last two babyscope (fluorescence) images denoted with 305b₀ and 305b-1 that have been acquired substantially at the same time as the motherscope images 305m₀ and 305m₋₁, respectively.

In the solution according to an embodiment of the present disclosure, a computing device receives both the motherscope images 305m and the babyscope images 305b; for example, the central unit of the babyscope receives the motherscope images 305m transmitted from the motherscope and the babyscope images 305b acquired by the babyscope (or vice-versa). Each (registration) pair of corresponding motherscope image 305m and babyscope image 305b is registered so as to bring the motherscope image 305m and the babyscope image 305b into spatial correspondence; for example, the motherscope image 305m and the babyscope image 305b are registered for correcting their misalignment due to the movement between the probe of the babyscope and the probe of the motherscope. This operation may be performed manually or automatically (as described in detail in the following). A representation of the body-part based on each pair of motherscope image 305m and babyscope image 305b so registered is then output, for example, the (registered) motherscope image 305m and babyscope image 305b are overlaid and displayed on a monitor coupled with the computing device (the monitor of the babyscope in this case).

As a result, it is possible to combine the fluorescence images of the babyscope (providing an accurate representation of the target of the medical procedure) with the reflectance images of the motherscope (providing a high-quality representation of the region of interest of the medical procedure). This significantly facilitates a task of a physician, thereby improving a result of the medical procedure with beneficial effects on the health of the patient.

In a specific implementation of the solution according to an embodiment of the present disclosure, for each registration pair of motherscope image 305m and babyscope image 305b, a (motherscope) motion of the motherscope is estimated according to the motherscope images 305m and a (babyscope) motion of the babyscope is estimated according to the babyscope images 305b, independently of each other; the motherscope motion is estimated according to a plurality of motherscope images 305m corresponding to the one to be registered (for example, the motherscope images 305m₀ and 305m₋₁ for the motherscope image 305m₀) and the babyscope motion is estimated according to a plurality of babyscope images 305b corresponding to the one to be registered (for example, the babyscope images 305b₀ and 305b-1 for the babyscope image 305b₀). For example, in the case of a simple translation as represented in the figure, the motherscope motion and the babyscope motion (direction and magnitude) are defined by a vector 310m and by a vector 310b, respectively.

The motherscope image 305m₀ and the babyscope image 305b₀ are then registered according to the motherscope motion 310m and the babyscope motion 310b. For example, a misalignment due to a relative movement between the motherscope and the babyscope is determined by a comparison between the motherscope motion 310m and the babyscope motion 310b. In the example at issue, a relative rotation of the babyscope with respect to the motherscope is defined by an angle 310mb equal to the difference between the vector 310b and the vector 310m; a correction 310bm given by the opposite of this angle 310mb is then applied to the babyscope image 305b₀ (by rotating it), so as to obtain a corresponding (registered) babyscope image 305b₀ᵣ that is now better aligned with the corresponding motherscope image 305m₀.

In this way, the babyscope images and the motherscope images are corrected continually so that in the end the effect of the relative movement between the babyscope and the motherscope is removed. As a result, the babyscope images and the motherscope images will be registered independently of their starting condition. In fact, the apparent motion of the fields of view of the motherscope and of the babyscope helps to estimate their relative position. For example, in the situation shown in the figure the translation of the babyscope with respect to the motherscope incrementally corrects their relative rotation.

More generally, the registration of the motherscope images 305m and the babyscope images 305b may involve any other transformation bringing them into spatial correspondence (i.e., referring them to a common reference space), for example, translation, rotation, zooming and so on. Particularly (not shown in the figure), it is possible to determine corresponding rotation center-points of the motherscope and of the babyscope, and then to calculate a relative translation of the babyscope with respect to the motherscope as defined by the distance from the rotation center-point of the motherscope to the rotation center-point of the babyscope; the babyscope image is then translated by the opposite of the resulting vector. As yet another example (again not shown in the figure), it is possible to determine corresponding zooming vector fields of the motherscope and of the babyscope, and then to calculate a magnification difference (zoom) of the babyscope with respect to the motherscope as defined by the difference between the zooming vector field of the motherscope and the zooming vector field of the babyscope; the babyscope image is then scaled by the opposite of the resulting magnification difference.

The above-described solution allows registering the babyscope images and the motherscope images automatically in an inherently robust way. In fact, the proposed solution does not involve any comparison between the babyscope images and the motherscope images. Therefore, an effective registration is then possible irrespectively of the fact that the babyscope/motherscope images have very limited and not easily distinguishable features (since mainly comprising smooth patches with low contrast that would be difficult to match in the different images with known feature-based methods). Moreover, the different characteristics of the babyscope images and of the motherscope images are completely immaterial to the obtained result. An effective registration is then possible even in case the babyscope images and the motherscope images are significantly different, especially when the babyscope only provides fluorescence images. In other words, rather than registering the babyscope images and the motherscope images directly, the desired result is achieved indirectly by working on the babyscope images and on the motherscope images separately. For this purpose, the unavoidable relative movements of the objects in the field of view of the babyscope and in the field of view of the motherscope is turned into an advantage; therefore, the cause itself of the misalignment between the babyscope images and the motherscope images (inherent movement between the babyscope and the motherscope) is exploited to remove (or at least reduce) the misalignment.

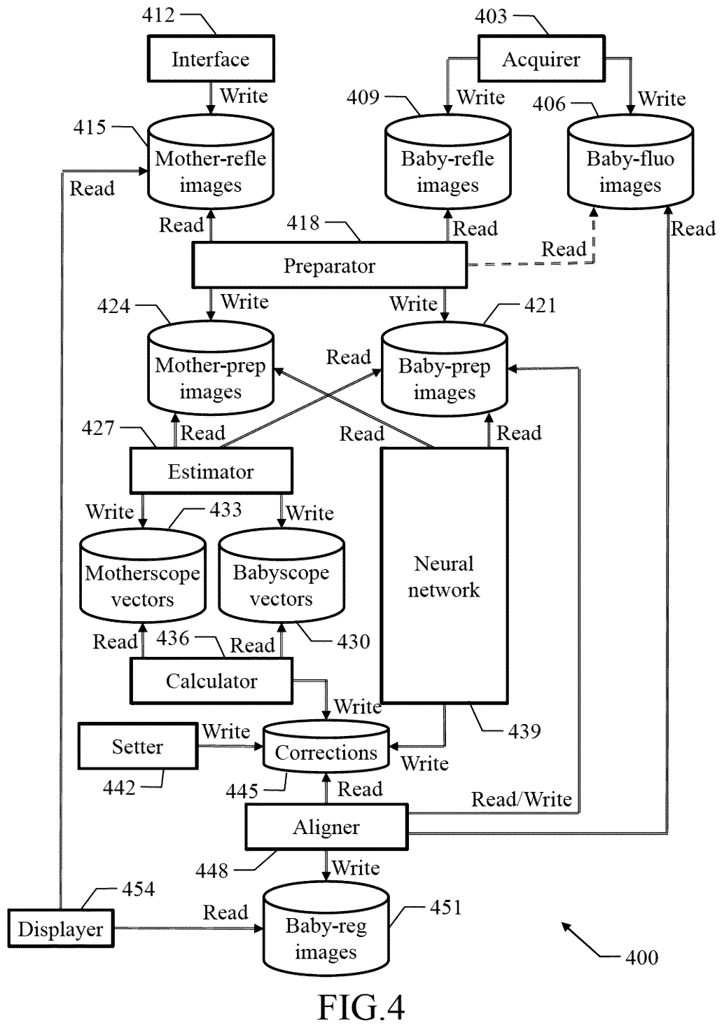

With reference now to FIG. 4, the main software components are shown that may be used to implement the solution according to an embodiment of the present disclosure.

All the software components (programs and data) are denoted as a whole with the reference 400. In a specific implementation, the software components 400 are stored in the mass memory and loaded (at least in part) into the working memory of the central unit of the babyscope when the programs are running, together with an operating system and other application programs not directly relevant to the solution of the present disclosure (thus omitted in the figure for the sake of simplicity). The programs are initially installed into the mass memory, for example, from removable storage units or from the network. In this respect, each program may be a module, segment or portion of code, which comprises one or more executable instructions for implementing the specified logical function.

Particularly, an acquirer 403 drives the components of the babyscope dedicated to acquiring the (babyscope) fluorescence images and the possible (babyscope) reflectance images of the field of view of the babyscope suitably illuminated for this purpose during a medical procedure. The acquirer 403 writes a (babyscope) fluorescence images repository 406 and a (babyscope) reflectance images repository 409, which contain corresponding sequences of the babyscope fluorescence images and of the babyscope reflectance images, respectively, that have been acquired in succession during the medical procedure. The babyscope fluorescence images repository 406 and the babyscope reflectance images repository 409 comprise corresponding entries for each pair of babyscope fluorescence image and babyscope reflectance image being acquired at a same acquisition time. The entry stores a bitmap of the corresponding babyscope (fluorescence/reflectance) image, which is defined by a matrix of cells (for example, with 512 rows and 512 columns) each containing a (color) value of a pixel, i.e., a basic picture element representing a corresponding location of the field of view of the babyscope; each pixel value of the babyscope fluorescence image defines the brightness of the pixel as a function of an intensity of the fluorescence light emitted by the location, whereas each pixel value of the babyscope reflectance image defines the brightness of the pixel as a function of an intensity of the visible light that is reflected by the location (for example, from 0 to 256 for each RGB component thereof). A video interface drive 412 drives the video interface of the babyscope; particularly, as far as relevant to the present disclosure, the video interface drive 412 receives the (motherscope) reflectance images that have been acquired by the motherscope during the medical procedure (from its video interface). The video interface drive 412 writes a (motherscope) reflectance images repository 415, which contains a sequence of the motherscope reflectance images that have been acquired in succession during the medical procedure by the motherscope substantially synchronously with the babyscope fluorescence/reflectance images. As above, the motherscope reflectance images repository 415 comprises an entry for each motherscope reflectance image. The entry stores a bitmap of the motherscope reflectance image, which is defined by a matrix of cells (typically with a higher resolution, for example, with 2048 rows and 2048 columns) each containing a (color) value of a pixel (representing a corresponding location of the field of view of the motherscope) that defines the brightness of the pixel as a function of an intensity of the visible light that is reflected by the location (for example, again from 0 to 256 for each RGB component thereof).

A preparator 418 prepares the (babyscope/motherscope) reflectance images by pre-processing them for their registration operation. The preparator 418 reads the babyscope reflectance images repository 409 and the motherscope reflectance images repository 415, and it writes a (babyscope) prepared images repository 421 and a (motherscope) prepared images repository 424. The babyscope prepared images repository 421 and the motherscope prepared images repository 424 comprise an entry for each babyscope reflectance image in the corresponding repository 409 and for each motherscope reflectance image in the corresponding repository 415, respectively; the entry stores a corresponding babyscope/motherscope prepared (reflectance) image. The babyscope/motherscope prepared image is formed by a matrix of cells (generally with a smaller size with respect to the corresponding babyscope/motherscope reflectance images), each storing a corresponding pixel value (for example, a grayscale value ranging from 0 for white to 256 for black). In case no babyscope reflectance images are available, the same operations described above are executed (in addition to the motherscope reflectance images) on the babyscope fluorescence images so as to obtain corresponding babyscope prepared (fluorescence) images. Therefore, in this case the preparator 418 reads the babyscope fluorescence images repository 406 (as shown in dashed line in the figure) instead of the babyscope reflectance images repository 409.

A corrector determines corrections (i.e., transformations) to be applied to the images provided by the babyscope and by the motherscope to register them automatically according to their motions estimated independently. The corrector may be implemented with an optical flow technique, with a deep learning technique or with a combination of both of them. The corrector based on the optical flow technique comprises an estimator 427, which estimates an optical flow of each babyscope/motherscope prepared image; the optical flow of the babyscope/motherscope prepared image represents an apparent motion of the content of the field of view of the babyscope/motherscope being due to its movement. The estimator 427 reads the babyscope prepared images repository 421 and the motherscope prepared images repository 424, and it writes a (babyscope) motion vectors repository 430 and a (motherscope) motion vectors repository 433. The babyscope motion vectors repository 430 and the motherscope motion vectors repository 433 comprise an entry for each babyscope/motherscope prepared image (different from the first one) in the corresponding repositories 421,424; the entry stores a corresponding babyscope/motherscope motion vector that defines the optical flow of the babyscope/motherscope prepared image (for example, a translation). The corrector further comprises a calculator 436 that calculates a correction for each (registration) pair of corresponding babyscope prepared image and motherscope prepared image according to the corresponding babyscope/motherscope motion vectors. The calculator 436 reads the babyscope motion vectors repository 430 and the motherscope motion vectors repository 433. Instead, the corrector based on the deep learning technique comprises a neural network 439, which directly determines the correction for each registration pair of babyscope prepared image and motherscope prepared image. The neural network 439 reads the babyscope prepared images repository 421 and the motherscope prepared images repository 424. A setter 442 is instead used to set the correction (to be applied to the images provided by the babyscope and by the motherscope to register them) manually. For this purpose, the setter 442 exposes a user interface providing one or more (software) dials (for example, rotational/linear sliders, input boxes, hotkeys and the like, designed to be controlled by the mouse, the keyboard and/or the trackball) and/or a drive for the setting device allowing entering the correction (such as rotation angle, translation direction and distance, zooming factor and so on). The calculator 436, the neural network 439 and the setter 442 write a corrections repository 445. Particularly, in case of the calculator 436 and the neural network 439 (automatic registration), the corrections repository 445 comprises an entry for each registration pair of babyscope/motherscope prepared images, which entry stores the corresponding correction, whereas in case of the setter 442 (manual registration), the corrections repository 445 stores the (common) correction for the (original) babyscope/motherscope images (in both cases, with each correction defined by a rotation angle in the example at issue).

An aligner 448 registers the images provided by the babyscope and by the motherscope according to the corresponding corrections. Particularly, the aligner 448 always registers each (registration) pair of corresponding babyscope fluorescence image and motherscope reflectance image according to the corresponding correction. For this purpose, the aligner 448 reads the babyscope fluorescence images repository 406 and the corrections repository 445, and it writes a (babyscope) registered fluorescence images repository 451. The babyscope registered fluorescence images repository 451 comprises an entry for each babyscope fluorescence image in the corresponding repository 406; the entry stores a corresponding (babyscope) registered fluorescence image. The babyscope registered fluorescence image is formed by a matrix of cells (with the same size as the babyscope fluorescence images), each storing a corresponding (color) pixel value. Moreover, in case the corrector is based on both the optical flow technique and the deep learning technique (to determine the corrections incrementally), the aligner 448 further (preliminary) registers each (registration) pair of corresponding babyscope prepared image and motherscope prepared image according to their (rough) correction. For this purpose, the aligner 448 further reads/writes the babyscope prepared images repository 421.

A displayer 454 drives the monitor of the babyscope for displaying a representation of the body-part based on the babyscope registered fluorescence images and the motherscope reflectance images (for example, overlaid to each other). The displayer 454 reads the babyscope registered fluorescence images repository 451 and the motherscope reflectance images repository 415.

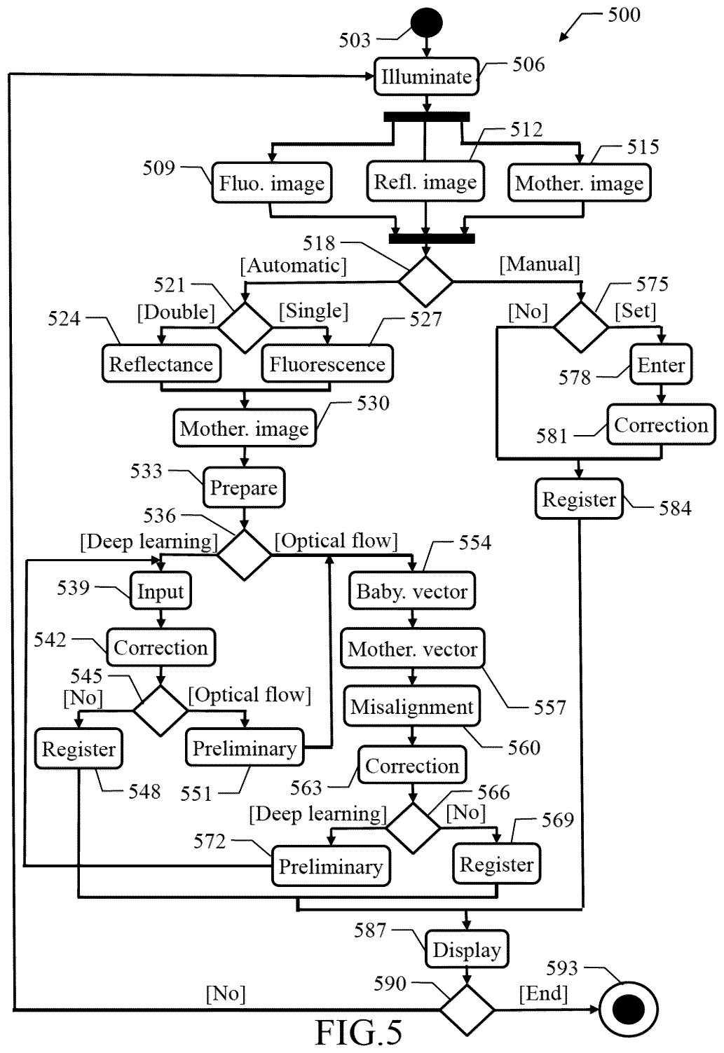

With reference now to FIG. 5, an activity diagram is shown describing the flow of activities relating to an implementation of the solution according to an embodiment of the present disclosure. In this respect, each block may correspond to one or more executable instructions for implementing the specified logical function on the central unit of the babyscope.

Particularly, the activity diagram represents an exemplary process that may be used for imaging a patient during a medical procedure with the above-described endoscope (endoscopic procedure) with a method 500.

Before the endoscopic procedure, a healthcare operator (for example, a nurse) administers a fluorescence agent to the patient. The fluorescence agent (for example, Indocyanine Green, Methylene Blue and so on) is adapted to reaching a specific (biological) target, such as a tumor to be inspected/resected/treated, and to remaining substantially immobilized therein. This result may be achieved by using either a non-targeted fluorescence agent (adapted to accumulating in the target without any specific interaction therewith, such as by passive accumulation) or a targeted fluorescence agent (adapted to attaching to the target by means of a specific interaction therewith, such as achieved by incorporating a target-specific ligand into the formulation of the fluorescence agent, for example, based on chemical binding properties and/or physical structures capable of interacting with different tissues, vascular properties, metabolic characteristics and so on). For instance, the fluorescence agent is administered to the patient intravenously as a bolus (for example, with a syringe); as a consequence, the fluorescence agent circulates within the vascular system of the patient until reaching the target and binding thereto; the remaining (unbound) fluorescence agent is instead cleared from the blood pool. After a waiting time allowing the fluorescence agent to accumulate in the (possible) tumor and to wash-out from the rest of the patient (for example, from some minutes to 24-72 hours), the endoscopic procedure may start. Therefore, if necessary a physician (completely or partially) anaesthetizes the patient. In any case, the physician inserts the probe of the motherscope, switched on by the (healthcare) operator, into the cavity of the patient (through the opening thereof) until its tip reaches the region of interest of the body-part wherein the tumor might be present. In response thereto, the white light source of the motherscope illuminates its field of view and the reflectance camera of the motherscope continually acquires motherscope reflectance images of its field of view that are displayed in real-time on the monitor of the motherscope (not shown in the figure).

As far as relevant to the solution according to an embodiment of the present disclosure, at a certain point of the endoscopic procedure the physician inserts the probe of the babyscope into a working channel of the motherscope (through the working port thereof) until its tip reaches the same region of interest of the body-part (at the tip of the probe of the motherscope). The physician may decide to switch on the babyscope and then start an imaging process (with the corresponding registration operation) at any time. For example, this may happen before inserting the babyscope into the working channel of the motherscope or later on once the tip of the probe of the babyscope has reached the tip of the probe of the motherscope. In any case, in response thereto the (imaging) process begins by passing from the black start circle 503 to block 506. At this point, the acquirer turns on the excitation light source and the white light source for illuminating the field of view of the babyscope. The flow of activity then forks into different operations that are performed concurrently. Particularly, the acquirer at block 509 acquires a new babyscope fluorescence image and adds it to the corresponding repository. Optionally, the acquirer at block 512 acquires a new babyscope reflectance image and adds it to the corresponding repository. Moreover, the video interface at block 515 (continually receiving the motherscope reflectance images that are transmitted from the motherscope) adds a last one of them to the corresponding repository. In this way, the babyscope fluorescence image and the babyscope reflectance image (when available) are acquired substantially at the same time and they provide different representations (in terms of fluorescence light and visible light, respectively) of the same field of view of the babyscope that are spatially coherent (i.e., a predictable correlation exists among their pixels, down to a perfect identity). Moreover, the motherscope reflectance image as well may be considered to have been acquired substantially at the same time, apart from a phase shift between the acquisition rates of the babyscope and of the motherscope (being negligible in practice).

The flow of activity joints again at block 518 from block 509, block 512 and block 515. At this point, the process branches according to a mode (manual/automatic) of the registration operation of the babyscope (for example, set manually, defined by default or the only one available). In case of an automatic mode of the registration operation, the process further branches at block 521 according to a structure of the babyscope. If the babyscope is structured to acquire both fluorescence images and reflectance images, the preparator at block 524 retrieves the (last) babyscope reflectance image just added to the corresponding repository. Conversely, if the babyscope is structured to acquire only fluorescence images, the preparator at block 527 retrieves the (last) babyscope fluorescence image just added to the corresponding repository. In both cases, the process continues to block 530 wherein the preparator retrieves the (last) motherscope reflectance image just added to the corresponding repository. The preparator at block 533 prepares the babyscope (reflectance or fluorescence) image and the motherscope (reflectance) image just retrieved for the corresponding registration operation. For example, the preparator may reduce each babyscope/motherscope image to a central region thereof by discarding its portion surrounding the central region, for example, defined by 15-25%, such as 20%, of its (outermost) pixels from each border of the babyscope/motherscope image. In this way, only the most useful part of the babyscope/motherscope images is taken into account, with corresponding increase of accuracy and reduction of computational time of the registration operation. In addition or in alternative, the preparator may downscale each (possibly reduced) babyscope/motherscope image, for example, by shrinking its size to 40-60%, such as 50% (for example, with low-pass filtering followed by sub-sampling). This reduces the computational time of the registration operation (for example, by 3-4 times), at the same time without adversely affecting its accuracy but rather slightly increasing it (by making minimal movements easier to detect). In addition or in alternative, the preparator may convert each (possibly reduced and/or downscaled) babyscope/motherscope image to grayscale, by replacing the RGB components of each pixel value with a single grayscale component representing a corresponding light intensity (such as from 0 for black to 255 for white), for example, with the grayscale component calculated as a weighted average of the RGB components (so as to preserve perceptual luminance). This reduces the computational time of the registration operation, at the same time increasing its accuracy and reducing a sensitivity to environment conditions (such as illumination, contrast, equipment). In any case, the preparator adds the babyscope/motherscope prepared images so obtained to the corresponding repositories. After a transient time, required to have a number of babyscope/motherscope prepared images enough for the registration operation (for example, 2-5), a (registration) pair of babyscope fluorescence image and motherscope reflectance image (as originally acquired) is registered. For this purpose, the flow of activity branches at block 536 according to a configuration of the babyscope (for example, set manually, defined by default or the only one available).

If the registration is based on the deep learning technique alone or preliminarily, the neural network is used to determine the corresponding correction. Basically, the neural network is a data processing system that approximates operation of human brain. The neural network comprises basic processing elements (neurons), which perform operations based on corresponding weights, the neurons are connected via unidirectional channels (synapses), which transfer data among them. The neurons are organized in layers performing different operations, always comprising an input layer and an output layer for receiving input data and for providing output data, respectively, of the neural network. In an embodiment of the present disclosure, the neural network is a Convolutional Neural Network (CNN), i.e., a specific type of deep neural network (with one or more hidden layers arranged in succession between the input layer and the output layer along a processing direction of the neural network) wherein one or more of its hidden layers perform (cross) convolution operations. For example, the neural network is based on a modified version of the VGG-16 model. More in detail, the input layer is configured to receive an input image that is generated by concatenating an (estimation) set of babyscope prepared images and an (estimation) set of prepared motherscope images, each estimation set comprising a plurality of the last babyscope/motherscope prepared images (for example, the last 2-10); the input image then has the same size as the babyscope/motherscope prepared images and a number of channels (values for each cell) given by the components of the pixel values of the babyscope/motherscope prepared images (for example, 4 channels for 2 babyscope prepared images and 2 motherscope prepared images each with 1 grayscale component for each pixel value). The hidden layers initially comprise 5 groups each of one or more convolutional layers (for example, a first group of 2 convolutional layers, a second group of 2 convolutional layers, a third group of 3 convolutional layers, a fourth group of 3 convolutional layers and a fifth group of 3 convolutional layers) followed by corresponding max-pooling layers In general, each convolutional layer performs a convolution operation through a convolution matrix (filter or kernel) defined by corresponding weights (for each channel, in the same numbers between the corresponding applied data and the filter), which convolution operation is performed in succession on limited portions of the applied data (receptive field) by shifting the filter across the applied data by a selected number of cells (stride), with the possible addition of cells with zero content around a border of the applied data (padding) to allow applying the filter thereto as well (to which a bias value may be added to translate it and then an activation function may be applied to introduce a non-linearity factor), so as to obtain corresponding filtered data. Every element of the filtered data of the convolutional layer may also be interpreted as an output of a neuron that looks at only a small region in the applied data (its receptive field) and shares parameters with other neurons to the left and to the right spatially (according to the stride of the filter that is shared among all the neurons of the convolutional layer). In the VGG-16 model, the convolutional layers apply a very small filter of 3×3, with a padding of 1 and a stride of 1 (so as to preserve a size of the applied data), with each neuron thereof applying a Rectified Linear Unit (ReLU) activation function. Each max-pooling layer is a pooling layer (down-sampling its applied data to reduce sensitivity to differences due to small displacements), which replaces the values of each limited portion of the applied data (window) with their maximum value, by shifting the window across the applied data by a selected number of cells (stride). In the VGG-16 model, the max-pooling layers have a windows of 2×2 with a stride of 2. As a whole, the above-mentioned groups of hidden layers decrease the size of the applied data down to 1×1 and increase theirs channels up to 4.096. The hidden layers then comprise 3 fully-connected (or dense) layers. Each fully-connected layer has its neurons that are connected to every neuron of the preceding layer. The fully-connected layers incrementally decrease the number of channels down to a number corresponding to all the possible classes of the input data. In the VGG-16 model, the fully-connected layers apply the ReLU activation function and decrease the channels to 1.000 for the ImageNet Large Scale Visual Recognition Challenge (ILSVRC) classification. In an embodiment of the present disclosure, the channels are reduced to provide 360 channels for the degrees of the rotation angle of the misalignment between a (registration) pair of the last babyscope prepared image and the last motherscope prepared image (from 0° to 359°); this is a good compromise between the opposed requirements of low complexity of the neural network (impacting its training and response time) and high resolution (impacting its accuracy). Moreover, in the first two fully-connected layers the ReLU activation function is replaced with a linear activation function. In the end, the hidden layers comprise a soft-max layer, which normalizes the applied data to a probability distribution (wherein each value ranges from 0 to 1 and all the values sum to 1). The output layer then determines the correction for the registration pair, set to the opposite of the rotation angle having the highest probability. In the implementation based on this deep learning technique, the process passes from block 536 to block 539, wherein the estimation set of babyscope images and the estimation set of motherscope images are concatenated into the input image of the neural network and applied thereto. Moving to block 542, the neural network directly outputs the correction for the corresponding registration pair of babyscope/motherscope prepared images, which correction is added to a value present in the last entry of the corresponding repository (initialized to 0 and possibly preliminary set with the optical flow technique as described below). As a result, when only the implementation based on the deep learning technique is applied, the correction determined with it directly defines the final value for the registration operation; conversely, when the implementation based on the optical flow technique has been applied previously, the correction determined with the deep learning technique refines its preliminary value determined with the optical flow technique. The flow of activity further branches at block 545 according to the configuration of the babyscope. If the correction so obtained is a final value to be applied to the registration pair of babyscope fluorescence image and motherscope reflectance image as originally acquired (implementation based on the optical flow technique not to be applied later on), the aligner at block 548 registers them accordingly; for example, the aligner applies the correction to the babyscope fluorescence image (by rotating it in the example at issue), and then adds the babyscope registered fluorescence image so obtained to the corresponding repository. The implementation based on the deep learning technique is very fast; therefore, it is well suited to real-time applications where the babyscope fluorescence images and the motherscope reflectance images are registered with a short delay from their acquisition so as to allow their display during the endoscopic procedure. Moreover, when the implementation based on the deep learning technique is applied after the implementation based on the optical flow technique, the neural network may be configured to provide a much lower number of values of the rotation angle (for example, 10 values from 0° to) 9°; in this case, the neural network may be trained more extensively, so as to be far more accurate. Referring back to block 545, if the correction so obtained is instead a preliminary value still to be refined by applying the optical flow technique, the aligner at block 551 likewise registers the registration pair of prepared babyscope/motherscope images by applying the correction to the prepared babyscope image and replacing it into the corresponding repository. The process then continues to block 554.

The same point is also reached directly from block 536 if the registration is based on the optical flow technique alone or preliminary. In both cases, the estimator now determines the babyscope motion vector (for example, a translation) of an (estimation) set of babyscope prepared images comprising a plurality of the last babyscope prepared images, for example, the last babyscope prepared image with respect to the last but one babyscope prepared image (extracted from the corresponding repository), and it adds the value so obtained to the corresponding repository. Likewise, the estimator at block 557 determines the motherscope motion vector (for example, again a translation) of an (estimation) set of motherscope prepared images comprising a plurality of the last motherscope prepared images, for example, the last motherscope prepared image with respect to the last but one motherscope prepared image (extracted from the corresponding repository), and it adds the value so obtained to the corresponding repository. For this purpose, the estimator may apply any known technique; for example, it is possible to calculate local motion vectors at the level of groups of one or more pixels (such as with a block-matching algorithm, or the estimation of a dense or sparse vector field), each of them representing an offset of the corresponding group of pixels from the last but one babyscope/motherscope prepared image to the last babyscope/motherscope prepared image; the (global) motion vector is then determined according to these local motion vectors (such as set to an average thereof). The calculator at block 560 calculates the misalignment (a rotation angle in the example at issue) between the registration pair of the last prepared babyscope/motherscope images according to the corresponding motion vectors (for example, equal to the angle from the motion vector of the babyscope prepared image to the motion vector of the motherscope prepared image) and adds it to the corresponding repository. The calculator at block 563 calculates the correction for the registration pair of babyscope/motherscope prepared images according to a (calculation) set of a plurality of last misalignments (for example, the last 2-10). Particularly, the correction is calculated as the opposite of a weighted average of the misalignments of the calculation set. For example, a weight is assigned to each misalignment decreasing (such as exponentially) with its age and/or extent. The weight decreasing with the age smooths the correction (by reducing the effect of jitters thereof) and the weight decreasing with the extent improves accuracy (by reducing the effect of misleading no, or very small, movements). The calculator then adds the correction so obtained to a value present in the last entry of the corresponding repository (initialized to 0 and possibly preliminary set with the deep learning technique as described above). As a result, when only the implementation based on the optical flow technique is applied, the correction determined with it directly defines the final value for the registration operation; conversely, when the implementation based on the deep learning technique has been applied previously, the correction determined with the optical flow technique refines its preliminary value determined with the deep learning technique. The flow of activity further branches at block 566 according to the configuration of the babyscope. If the correction so obtained is a final value to be applied to the registration pair of babyscope fluorescence image and motherscope reflectance image as originally acquired (implementation based on the deep learning technique not to be applied later on), the aligner at block 569 registers the registration pair of babyscope fluorescence image and motherscope reflectance image (as originally acquired) by applying the correction to the babyscope fluorescence image and adding the babyscope registered fluorescence image so obtained to the corresponding repository. The implementation based on the motion flow technique is very accurate; therefore, it is well suited to off-line applications (wherein the required computational time is immaterial) or to real-time applications for refining a preliminary registration provided by the implementation based on the deep learning technique (wherein the latter significantly reduces its computational time). Referring back to block 566, if the correction so obtained is instead a preliminary value still to be refined by applying the deep learning technique, the aligner at block 572 likewise registers the registration pair of prepared babyscope/motherscope images by applying the correction to the prepared babyscope image and replacing it into the corresponding repository. The process then returns to block 539.

Referring back to block 518, in case of a manual mode of the registration operation, the setter at block 575 verifies whether the correction (to be applied to the registration pairs of babyscope/motherscope images as originally acquired) needs to be set. Particularly, this always happens at the beginning of the endoscopic procedure (for initializing the correction) and possibly during it when the physician decides that the correction is not accurate any longer (for updating the correction). If the correction has to be set, the process descends into block 578, wherein the physician enters the correction via the setter. For example, the displayer continuously displays the motherscope reflectance images and the babyscope reflectance images together on the monitor of the babyscope (the same operations are performed using the babyscope fluorescence images when the babyscope reflectance images are not available). The physician waits until a (relatively) stationary condition of the endoscopic procedure has been reached (as shown by the displayed motherscope/babyscope reflectance images). For example, this happens when the tip of the probe of the motherscope has reached the region of interest of the body-part and the probe of the babyscope has been inserted into the working channel of the motherscope with its tip that has reached the same region of interest of the body-part. The physician may now act on the motherscope/babyscope reflectance images (either via the user interface or the setting device) until they appear to be registered. This operation may be performed on the motherscope/babyscope reflectance images while played during their acquisition or paused in response to a corresponding command entered by the physician into the babyscope (for example, with its keyboard). Once the operation has been completed, the physician confirms the (manual) registration of the motherscope/babyscope reflectance images so obtained by entering a corresponding command into the babyscope (for example, with its keyboard). In response thereto, the setter at block 581 determines the correction corresponding to this manual registration, and saves it into the corresponding repository (replacing its previous value, initialized to null). The process then descends into block 584; the same point is also reached directly from block 575 when no setting of the correction is required. At this point, the aligner registers the registration pair of babyscope fluorescence image and motherscope reflectance image (as originally acquired) by applying the correction (extracted from the corresponding repository) to the babyscope fluorescence image and adding the babyscope registered fluorescence image so obtained to the corresponding repository.

The flow of activity then merges again at block 587 from block 548 (automatic mode based on the deep learning technique alone or additionally), from block 569 (automatic mode based on the optical flow technique alone or additionally) or from block 584 (manual mode). At this point, the displayer displays a representation of the body-part based on the motherscope reflectance image and the babyscope registered fluorescence image. For example, the displayer may display an overlaid image that is created by overlaying the babyscope registered fluorescence image onto the motherscope reflectance image (with each pixel value of the overlaid image equal to the corresponding pixel value of the babyscope registered fluorescence image when its brightness is, possibly strictly, higher than a threshold, such as 5-10% of its maximum value, or equal to the corresponding pixel value of the motherscope reflectance image otherwise).

With reference now to block 590, if the imaging process is still in progress, the flow of activity returns before blocks 509-515 to repeat the same operations continually Conversely, if the imaging process has ended, as indicated by an end command entered into the babyscope by the operator (for example, with its keyboard), the process ends at the concentric white/black stop circles 593 (after turning off the excitation light source and the white light source by the acquirer).

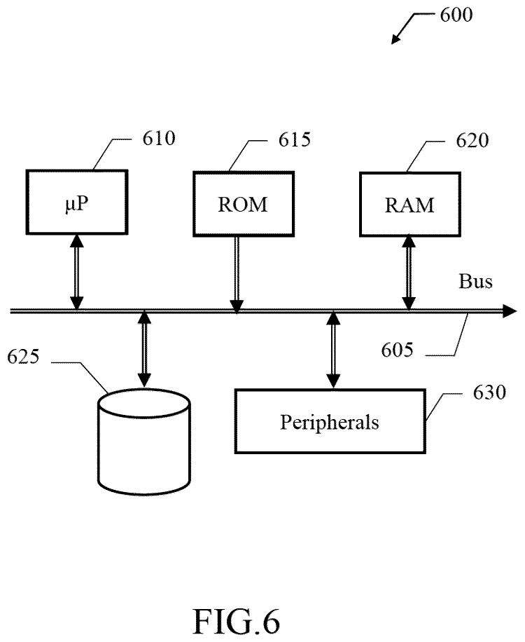

With reference now to FIG. 6, a schematic block diagram is shown of a (training) computing system 600 that may be used to train the neural network of the solution according to an embodiment of the present disclosure.

The training (computing) system 600, for example, a Personal Computer (PC), comprises several units that are connected among them through a bus structure 605. Particularly, a microprocessor (μP) 610, or more, provides a logic capability of the training system 600. A non-volatile memory (ROM) 615 stores basic code for a bootstrap of the training system 600 and a volatile memory (RAM) 620 is used as a working memory by the microprocessor 610. The training system 600 is provided with a mass-memory 625 for storing programs and data, for example, a Solid-State-Disk (SSD). Moreover, the training system 600 comprises a number of controllers 630 for peripherals, or Input/Output (I/O) units; for the example, the peripherals comprise a keyboard, a mouse, a monitor, a network adapter (NIC) for connecting to a communication network (such as the Internet), a drive for reading/writing removable storage units (such as of USB type) and so on.

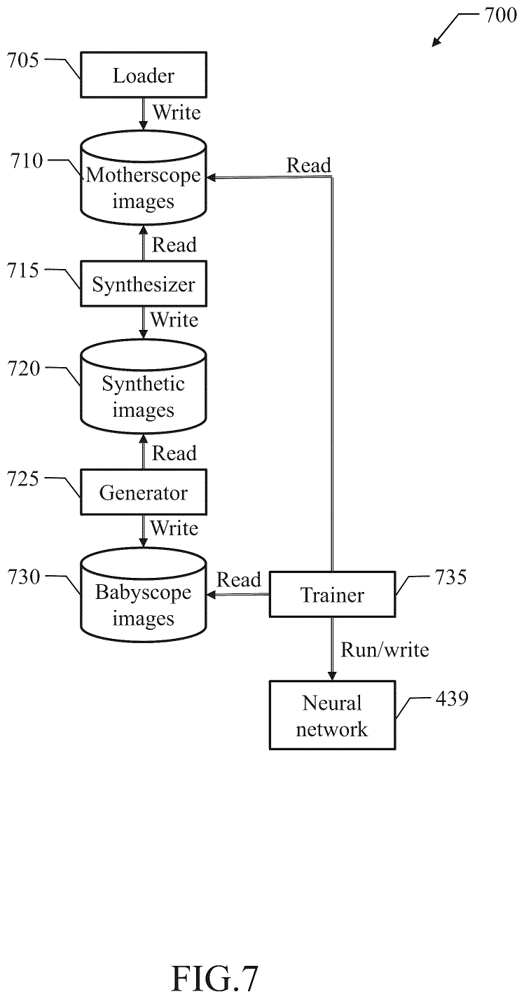

With reference now to FIG. 7, the main software components are shown that may be used to train the neural network in the solution according to an embodiment of the present disclosure.

All the software components (programs and data) are denoted as a whole with the reference 700. The software components 700 are stored in the mass memory and loaded (at least in part) into the working memory of the training system when the programs are running, together with an operating system and other application programs not directly relevant to the solution of the present disclosure (thus omitted in the figure for the sake of simplicity). The programs are initially installed into the mass memory, for example, from removable storage units or from the network. In this respect, each program may be a module, segment or portion of code, which comprises one or more executable instructions for implementing the specified logical function.

A loader 705 loads a plurality of sample motherscope (reflectance) images that have been acquired as above during a sample endoscopic procedure, or more, with a corresponding sample motherscope (for example, by imaging a colon of a patient with a tumor); if possible, the (sample) motherscope images are acquired with different models of the sample motherscope, to make the neural network less sensitive to the actual model of motherscope used in practice. The loader 705 writes a (sample) motherscope images repository 710, which stores the motherscope images. A synthesizer 715 synthesizes corresponding (babyscope) synthetic images from the motherscope images, which synthetic images mimic corresponding reflectance images as acquired by a babyscope, or more, inserted through a working channel of the sample motherscope. The synthesizer 715 reads the motherscope images repository 710, and it writes a synthetic images repository 720, which stores the synthetic images. A generator 725 generates a plurality of (sample) babyscope (reflectance) images from the synthetic images by applying different misalignments thereto (for example, multiple rotations selected randomly to each of them). The generator 725 reads the synthetic images repository 720, and it writes a (sample) babyscope images repository 730. The babyscope images repository 730 comprises an entry for each babyscope image; the entry stores the babyscope image, an indication of the motherscope image used to synthesize the corresponding synthetic image (for example, its index in the corresponding repository 710) and a (reference) correction equal to the opposite of the misalignment used to generate the babyscope image (then representing its gold value). A trainer 735 trains the neural network 439. The trainer 735 reads the motherscope images repository 710 and the babyscope images repository 730; moreover, the trainer 735 runs a copy of the neural network, denoted with the same reference 439 (applying the input data and receiving the output data) and writes its weights.

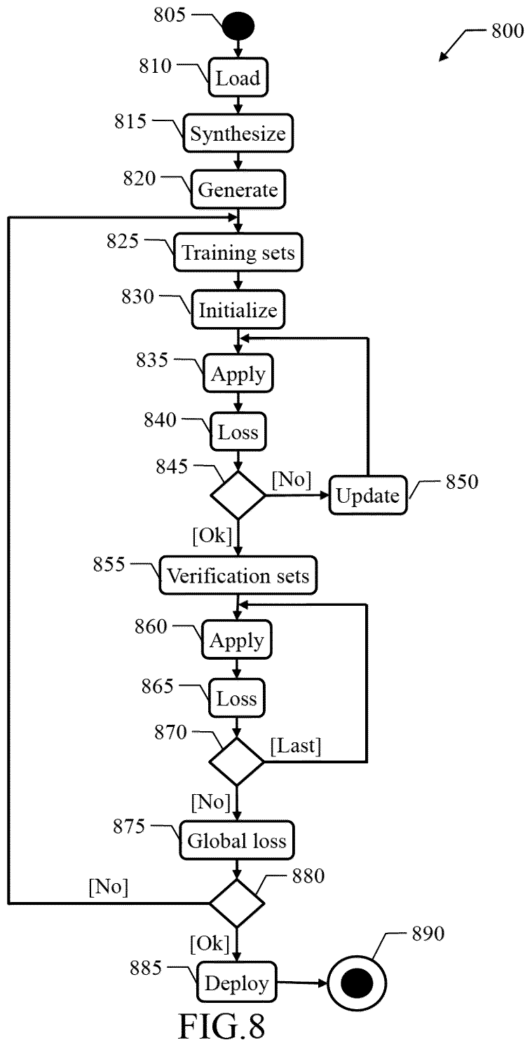

With reference now to FIG. 8, an activity diagram is shown describing the flow of activities relating to the training of the neural network in the solution according to an embodiment of the present disclosure.

Particularly, the activity diagram represents an exemplary process that may be used to train the neural network with a method 800 (during a development of the babyscope and possibly during next maintenance thereof). As above, each block may correspond to one or more executable instructions for implementing the specified logical function on the training system.

The process begins at the black start circle 805 and then passes to block 810, wherein the trainer loads (for example, via a removable storage unit or the network) the motherscope images into the corresponding repository. The synthesizer at bock 815 synthesizes the corresponding synthetic image from each motherscope image (retrieved from the corresponding repository); for this purpose, the synthesizer applies a number of updates to the motherscope image aimed at making it resemble a realistic reflectance image acquired with a babyscope. Particularly, the motherscope image (generally having a polygonal, such as rectangular or octagonal, shape) is converted to a circular shape (for example, by resetting to zero the values of the pixels outside the smaller circle inscribed in the motherscope image). In addition or in alternative, a resolution of the motherscope image is reduced (for example, by down-sampling it by 30-70%, such as by 50%). In addition or in alternative, a contrast of the motherscope is reduced (for example, by applying a low contrast filter). In addition or in alternative, the motherscope image is zoomed in to simulate a smaller field of view (for example, by replicating the values of the pixels in a central zone). In addition or in alternative, the motherscope image is translated (for example, by a random value). In addition or in alternative, noise is added to the motherscope image (for example, by adding random noise to its pixel values). The synthesizer adds the synthetic images so obtained to the corresponding repository (at the same positions of the corresponding motherscope images in their repository so as to associate them). The generator at block 820 generates the babyscope images from the synthetic images. Particularly, for each synthetic image (retrieved from the corresponding repository) the generator produces a plurality of pseudo-random values of a rotation angle (for example, 100-400) and then applies them to the synthetic image. The generator adds each babyscope image so obtained to the corresponding repository, together with the indication of the corresponding motherscope image (for example, equal to a common index of the synthetic/motherscope images in their repositories) and the corresponding reference correction (equal to the opposite value of the applied rotation angle).

The trainer now performs a training operation of the neural network to find (optimized) values of its weights, and possibly its parameters, that optimize performance. For this purpose, the trainer at block 825 selects a plurality of training sets, each one formed by a babyscope image, the corresponding motherscope image and the corresponding reference correction. The training sets are defined by sampling the babyscope images to select a percentage thereof (for example, 50% selected randomly). The trainer at block 830 initializes the weights of the neural network randomly. A loop is then entered at block 835, wherein the trainer feeds the babyscope image of each training set in succession (in any arbitrary order) to the neural network to obtain the corresponding (estimated) correction. The trainer at block 840 calculates a loss value based on a difference between the estimated correction and the reference correction of the training set (for example, by applying the Huber function to limit sensitivity to outliers). The trainer at block 845 verifies whether the loss value is not acceptable and it is still improving significantly. This operation may be performed either in an iterative mode (after processing each training set for its loss value) or in a batch mode (after processing all the training sets for a cumulative value of their loss values, such as an average thereof). If so, the trainer at block 850 updates the weights of the neural network in an attempt to improve performance of the neural network. For example, in a process based on the Stochastic Gradient Descent (SGD) algorithm, a direction and an amount of the change is given by a gradient of a loss function expressing the loss value as a function of the weights being approximated with a backpropagation algorithm. The process then returns to block 835 to repeat the same operations. With reference again to block 845, if the loss value has become acceptable or the change of the weights does not provide any significant improvement (meaning that a minimum, at least local, or a flat region of the loss function has been found) the loop is exited. The above-described loop may be performed by adding a random noise to the weights and/or it may be reiterated starting from different initializations of the neural network to find different (and possibly better) local minimums and to discriminate the flat regions of the loss function.

Once a configuration of the neural network has been found providing an optimal minimum of the loss function, the process continues to block 855. At this point, the trainer performs a verification operation of the performance of the neural network so obtained. For this purpose, the trainer selects a plurality of verification sets, each one formed by a babyscope image, the corresponding motherscope image and the corresponding reference correction. For example, the verification sets are defined by the babyscope images different from the ones of the training sets. A loop is then entered at block 860, wherein the trainer feeds the babyscope image of a (current) verification set (starting from a first one in any arbitrary order) to the neural network to obtain the corresponding (estimated) correction. The trainer at block 865 calculates the loss value as above based on the difference between the estimated correction and the reference correction of the verification set. The trainer at block 870 verifies whether a last verification set has been processed. If not, the flow of activity returns to block 860 to repeat the same operations on a next verification set. Conversely (once all the verification sets have been processed) the loop is exit by descending into block 875. At this point, the trainer determines a global loss of the above-mentioned verification (for example, equal to an average of the loss values of all the verification sets). The flow of activity branches at block 880 according to the global loss. If the global loss is (possibly strictly) higher than an acceptable value, this means that the capability of generalization of the neural network (from its configuration learned from the training sets to the verification sets) is too poor; in this case, the process returns to block 825 to repeat the same operations with different training sets, training parameters (such as learning rate, epochs number and so on) and/or neural network parameters (such as number of correction values, activation functions and so on), or to block 820 to increase the babyscope images (not shown in the figure). Conversely, if the global loss is (possibly strictly) lower than the acceptable value, this means that the capability of generalization of the neural network is satisfactory; in this case, the trainer at block 885 accepts the configuration of the neural network for its deployment to a batch of instances of the babyscope. The process then ends at the concentric white/black stop circles 890.

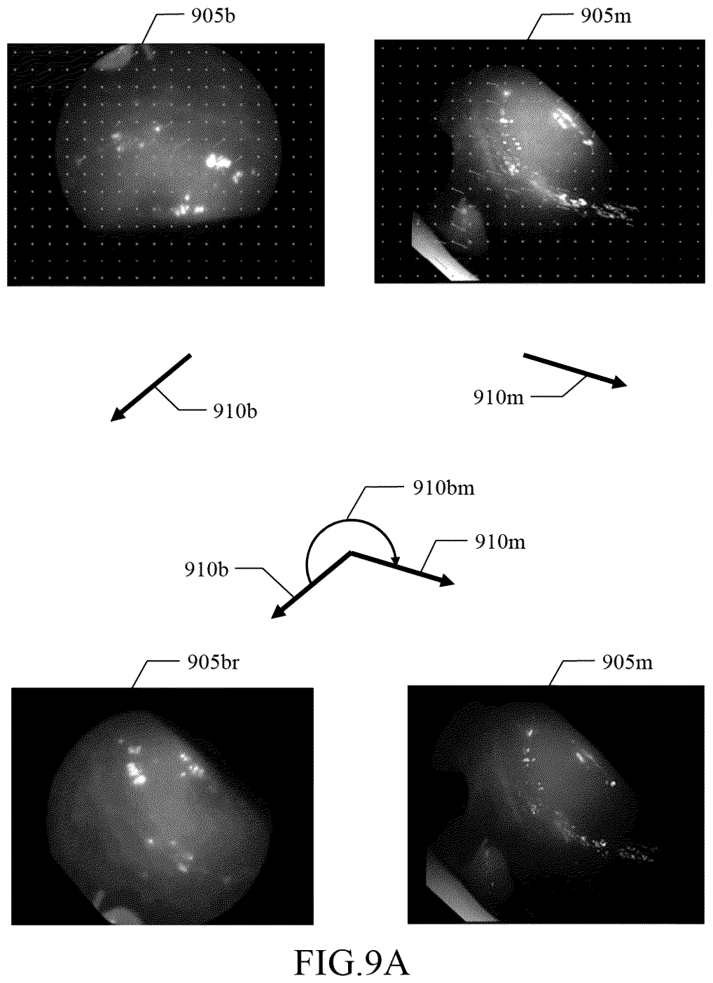

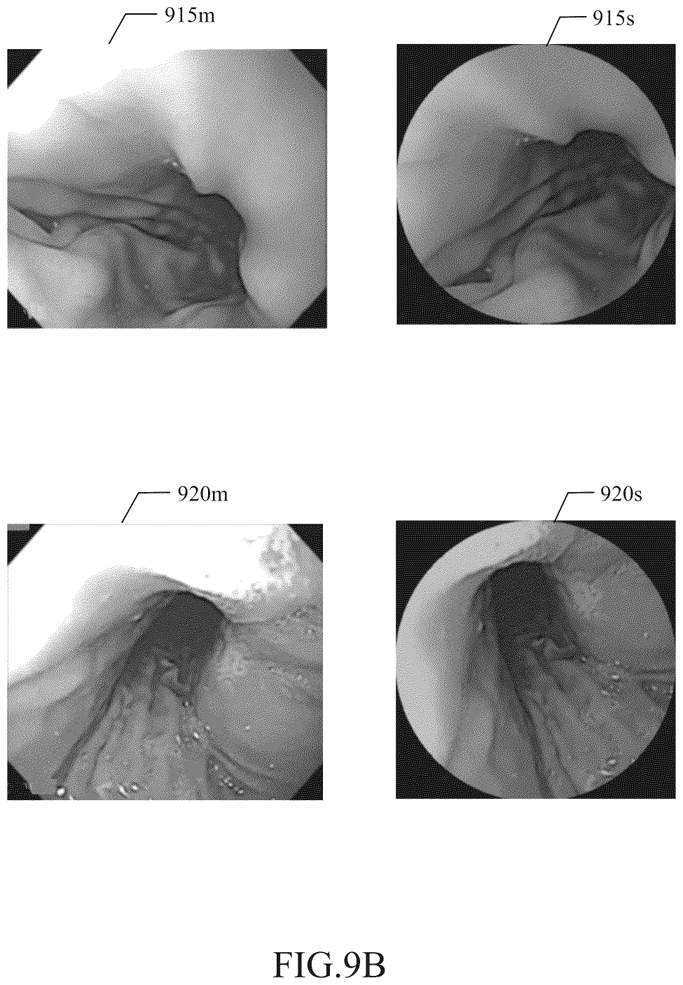

With reference now to FIG. 9A-FIG. 9B, different examples are shown of application of the solution according to an embodiment of the present disclosure.

Starting from FIG. 9A, it relates to an exemplary registration during an endoscopic procedure. Particularly, the figure shows a babyscope (reflectance) image 905b and a motherscope (reflectance) image 905m of a registration pair, with the addition of the corresponding local motion vectors. A babyscope motion vector 910b and a motherscope motion vector 910m are determined for the babyscope image 905b and for the motherscope image 905m, respectively (from their local motion vectors). A correction (rotation angle) 910bm is calculated as the motherscope motion vector 910m minus the babyscope motion vector 910b. The babyscope image 905b is then registered according to the correction 910bm (rotating by the corresponding angle), so as to obtain a corresponding babyscope registered (fluorescence) image 910br; as can be seen, the babyscope registered image 910br is now substantially aligned with the motherscope image 905m (shown again without the local motion vectors).

Moving to FIG. 9B, it relates to an exemplary training of the neural network. Particularly, the figure shows two motherscope (reflectance) images 915m and 920m that have been acquired during a sample endoscopic procedure. Two synthetic (reflectance) images 915s and 920s (mimicking corresponding babyscope reflectance images) are synthesized from the motherscope images 915m and 920m, respectively (by converting them to a circular shape, reducing resolution/contrast and zooming in).

### Modifications

In order to satisfy local and specific requirements, a person skilled in the art may apply many logical and/or physical modifications and alterations to the present disclosure. More specifically, although this disclosure has been described with a certain degree of particularity with reference to one or more embodiments thereof, it should be understood that various omissions, substitutions and changes in the form and details as well as other embodiments are possible. Particularly, different embodiments of the present disclosure may be practiced even without the specific details (such as the numerical values) set forth in the preceding description to provide a more thorough understanding thereof; conversely, well-known features may have been omitted or simplified in order not to obscure the description with unnecessary particulars. Moreover, it is expressly intended that specific elements and/or method steps described in connection with any embodiment of the present disclosure may be incorporated in any other embodiment as a matter of general design choice. Moreover, items presented in a same group and different embodiments, examples or alternatives are not to be construed as de facto equivalent to each other (but they are separate and autonomous entities) In any case, each numerical value should be read as modified according to applicable tolerances; particularly, unless otherwise indicated, the terms “substantially”, “about”, “approximately” and the like should be understood as within 10%, preferably 5% and still more preferably 1%. Moreover, each range of numerical values should be intended as expressly specifying any possible number along the continuum within the range (comprising its end points). Ordinal or other qualifiers are merely used as labels to distinguish elements with the same name but do not by themselves connote any priority, precedence or order. The terms include, comprise, have, contain, involve and the like should be intended with an open, non-exhaustive meaning (i.e., not limited to the recited items), the terms based on, dependent on, according to, function of and the like should be intended as a non-exclusive relationship (i.e., with possible further variables involved), the term a/an should be intended as one or more items (unless expressly indicated otherwise), and the term means for (or any means-plus-function formulation) should be intended as any structure adapted or configured for carrying out the relevant function.

For example, an embodiment provides a method for imaging a body-part of a patient with an endoscopic system. However, the (imaging) method may be used for imaging any body-part (for example, one or more organs, a region, tissues and the like of the gastrointestinal tract, respiratory tract, urinary tract, uterus, internal joint and so on) and of any patient (for example, a human being, an animal and so on) with any endoscopic system (see below); moreover, the method may be used in any medical procedure (for example, surgery, diagnostics, therapy and so on). The method only relates to operations for controlling the endoscopic system, independently of any interaction with the patient (or at most without any substantial physical intervention on the patient that would require professional medical expertise or entail any health risk for him/her). In any case, although the method may facilitate the task of a physician, it only provides intermediate results that may help him/her but with the medical activity stricto sensu that is always made by the physician himself/herself.

In an embodiment, the method comprises acquiring (with a first endoscopic unit of the endoscopic system) a first sequence of a plurality of first images of a first field of view containing at least part of the body-part. However, the first images may be in any number and of any type (for example, reflectance images, luminescence images, ultrasound images, representing the whole body-part or only a portion thereof, and so on), and they may be acquired with any first endoscopic unit (see below).

In an embodiment, the first sequence of first images is acquired via a first probe of the first endoscopic unit. However, the first probe may be of any type (for example, based on fiber bundle, chip-on tip and so on).

In an embodiment, the method comprises acquiring (with a second endoscopic unit of the endoscopic system) a second sequence of a plurality of second image sets corresponding to the first images. However, the first images and the second image sets may correspond in any way (for example, by acquiring them independently, with different start times and frequencies, and then associating each image of a sequence with the last image of the other sequence being available or with the preceding/following image of the other sequence being closer thereto, by acquiring the first images and the second images in synchrony, by receiving the images of a sequence, determining its phase and frequency, and then starting acquiring the images of the other sequence in synchrony, and so on), and the second images may be acquired with any second endoscopic unit (see below).

In an embodiment, each of the second image sets comprises one or more second images of a second field of view containing at least part of the body-part. However, each second image set may comprise any number of second images of any type (for example, luminescence images, reflectance images, ultrasound images, any combination thereof, representing the whole body-part or only a portion thereof, and so on). The first field of view and the second field of view may be of any type (for example, different from each other, such as overlapping to any extent or disjoint, such as to image a thinner hollow organ that branches off a ticker hollow organ, like in choledochoscopy, equal to each other, and so on).

In an embodiment, the second sequence of second images is acquired via a second probe of the second endoscopic unit that is movable with respect to the first probe. However, the second probe may be of any type (for example, based on chip-on tip, fiber bundle and so on); moreover, the two probes may be movable between them in any way that is not known a priori (for example, by rotating, translating, any combination thereof, always or only before a coupling thereof, and so on).

In an embodiment, the method comprises registering (by a computing device) each registration pair of a first image of the first images and a second image of the corresponding second image set. However, the registration may be of any type (for example, any affine transformation such as rotation, translation, both of them and the like, a non-rigid transformation, such as zooming, warping, and so on); moreover, the registration may be performed in any way (for example, automatically, semi-automatically or manually, by updating the first image only, the second image only or both of them, and so on) and at any time (for example, by determining a correction to be applied continually when the two probes are always movable between them, only at the coupling of the two probes when they are fixed to each other after that and so on) by any computing device (see below).

In an embodiment, the method comprises outputting (on an output unit) a representation of the body-part based on the first image and the second image of each registration pair being registered. However, the representation of the body-part may be output in any way (for example, displayed, printed, transmitted remotely, in real-time or off-line, and so on) on any output unit (see below); moreover, the representation of the body part may be based on the registered images in any way (for example, by outputting the images of each registration pair overlaid, side-by-side and so on).

Further embodiments provide additional advantageous features, which may however be omitted at all in a basic implementation.

Particularly, in an embodiment the method comprises acquiring (with the second endoscopic unit) the second sequence of second image sets via the second probe inserted removably into a working channel of the first endoscopic unit. However, the second probe may be inserted into the working channel in any way (for example, either after or before inserting the motherscope into the cavity, and so on). In any case, the possibility is not excluded of having the motherscope and the babyscope arranged in any other way (for example, inserted independently into the cavity, with the babyscope inserted into a trocar port of a laparoscope, and so on).

In an embodiment, the computing device is comprised in one between the first endoscopic unit and the second endoscopic unit. However, the computing device may be comprised in any one of the endoscopic units.

In an embodiment, the method comprises receiving (by the computing device) the corresponding first sequence of first images or second sequence of second image sets from the other one of the first endoscopic unit and the second endoscopic unit. However, these (first or second) images may be received in any way (for example, in push mode, in pull mode, over any wired/wireless communication channel, from a removable storage unit, downloaded from a network and so on).

In an embodiment, the method comprises determining (by the computing device) at least one correction for each registration pair of first image and second image due to a corresponding relative movement of the first probe and the second probe. However, the correction may be of any type (for example, a single correction value for the first image or the second image of the registration pair, two corresponding correction values for the first image and the second image of the registration pair, and so on) and it may be determined in any way (for example, only with an optical flow technique, only with a deep learning technique, with both of them in any order, manually and so on).

In an embodiment, the method comprises registering (by the computing device) each registration pair of first image and second image according to the corresponding correction. However, the registration pair of first image and second image may be registered according to the correction in any way (for example, by applying it completely, incrementally and so on).

In an embodiment, the method comprises estimating (by the computing device) a first motion of the first probe and a second motion of the second probe independently for each current one of the first images and for a current second image of each current one of the second image sets, respectively, the first motion being estimated according to an estimation set of a plurality of the first images corresponding to the current first image and the second motion being estimated according to an estimation set of a plurality of the second images corresponding to the current second image. However, the motions may be of any type (for example, rotations, translations, both of them and so on) and indicated in any way (for example, by motion vectors, rotation center-points, zooming vector fields and so on); the motions may be estimated in any way (for example, with optical flow technique, deep learning technique, any combination thereof and so on) according to any type of estimation set of first/second images (for example, comprising any number of images, selected among all the preceding ones or with some temporal subsampling, and so on).

In an embodiment, the method comprises determining (by the computing device) the correction for each registration pair of first image and second image according to the first motions and the second motions of a calculation set of one or more of the first images and of the second images, respectively, corresponding to the pair of first image and second image. However, the correction may be determined in any way (for example, by calculating a misalignment between each pair of corresponding first image and second images and then the correction from the misalignments of the calculation set, calculating a first displacement and a second displacement from the first motions and the second motions, respectively, of the calculation set and then the correction from the first displacement and the second displacement, and so on) according to the first/second motions of the calculation set (for example, as the average, median, mode and the like of the relevant values being weighted in any way or even as is, and so on).

In an embodiment, the method comprises estimating (by the computing device) a first motion vector indicative of the first motion for each current first image according to the corresponding estimation set of first images and a second motion vector indicative of the second motion for each current second image according to the corresponding estimation set of second images. However, the motion vectors may be of any type (for example, dense vector field with two components for each location, sparce vector field with vector components for some of the locations, and so on) and they may be estimated in any way (for example, by applying block-matching, phase correlation, differential and so on methods, by aggregating local values for the locations or groups thereof in any way, such as according to their average, dominant motions and the like, directly at the level of the whole images and so on).

In an embodiment, the method comprises calculating (by the computing device) a misalignment for each pair of corresponding current first image and current second image according to the corresponding first motion and second motion.

However, the misalignment may be calculated in any way (for example, as difference of motion vectors, distance of rotation center-points, difference between zooming vector fields and so on).

In an embodiment, the method comprises calculating (by the computing device) the correction for each registration pair of first image and second image according to the misalignments of the corresponding calculation set of first images and second images. However, the correction may be calculated in any way according to the misalignments (for example, as the average, median, mode and the like of the misalignments being weighted in any way or even as is, and so on).

In an embodiment, for this purpose the misalignments are weighted decreasingly with corresponding extents. However, the misalignments may be weighed according to their extents in any way (for example, according to a continuous or a discrete function, exponentially, linearly and the like, by simply disregarding the ones below a threshold and so on).

In an embodiment, for this purpose the misalignments are weighted decreasingly with corresponding temporal distances from the registration pair of first image and second image. However, the misalignments may be weighed according to their temporal distances in any way (for example, either the same or different with respect to above).

In an embodiment, the method comprises supplying (by the computing device) a neural network with input data based on a further estimation set of first images and a further estimation set of second images for each registration pair of first image and second image. However, the neural network may be of any type (for example, a convolutional neural network, a time delay neural network, a recurrent neural network, a modular neural network and so on) and it may be supplied with input data based on any number of first/second images in any way (for example, a common concatenation of the first images and the second images, a concatenation of the first images and a concatenation of the second images, the first/second images separately and so on).

In an embodiment, the method comprises receiving (by the computing device) the correction for each registration pair of first image and second image from the neural network. However, the neural network may provide the correction in any way (for example, defining a classification result, a regression result and so on).

In an embodiment, the method comprises selecting (by the neural network) the correction for each registration pair of first image and second image among a plurality of pre-defined corrections. However, the pre-defined corrections may be in any number and of any type (for example, distributed uniformly, more concentrated in certain ranges and so on).

In an embodiment, the neural network is a convolutional neural network. However, the convolutional neural network may be of any type (for example, derived from a known model, such as VGG-16, VGG-19, ResNet50 and the like, custom and so on).

In an embodiment, the convolutional neural network comprises (along a processing direction of the convolutional neural network) a plurality of groups, each comprising one or more convolutional layers followed by a max-pooling layer. However, the groups may be in any number each comprising any number and type of convolutional layers followed by any type of max-pooling layer (for example, with any receptive field, stride, padding, activation function and so on).

In an embodiment, the convolutional neural network comprises (along the processing direction of the convolutional neural network) a plurality of fully connected layers providing corresponding probabilities of the pre-defined corrections. However, the fully-connected layers may be in any number and of any type (for example, with any number of channels, activation functions and so on).

In an embodiment, the method comprises acquiring (with the first endoscopic unit) the first sequence of first images each comprising a plurality of first values representative of corresponding locations of the first field of view. However, each first value may comprise any number and type of components (for example, RBG, XYZ, CMYK, grey-scale and so on).

In an embodiment, the method comprises acquiring (with the second endoscopic unit) the second sequence of second image sets with the second images each comprising a plurality of second values representative of corresponding locations of the second field of view. However, each second value may comprise any number and type of components (for example, either the same or different with respect to the first components).

In an embodiment, the method comprises supplying (by the computing device) the neural network with the input data for each registration pair of first image and second image being obtained by concatenating the first values of the first images of the further estimation set and the second values of the second images of the further estimation set for each location. However, the images may be concatenated in any way (for example, the first images followed by the second images, vice-versa and so on).

In an embodiment, the method comprises receiving (by the computing device) the correction being entered manually. However, the correction may be entered in any way (for example, via partial, different and/or additional software and/or hardware commands with respect to the ones mentioned above).

In an embodiment, the method comprises acquiring (with the first endoscopic unit) the first sequence of first images being corresponding reflectance images representative of a visible light reflected by a content of the first field of view. However, the reflectance images may be of any type (for example, in colors, black-and-white, hyper-spectral and so on).

In an embodiment, the method comprises acquiring (with the second endoscopic unit) the second sequence of second image sets being corresponding luminescence images (representative of a luminescence light emitted in the second field of view by a luminescence substance) and corresponding further reflectance images (representative of the visible light reflected by a content of the second field of view). However, the luminescence light may be of any type (for example, NIR, Infra-Red (IR), visible and so on) and it may be emitted in any way (for example, in response to a corresponding excitation light or more generally to any other excitation different from heating) by any extrinsic/intrinsic or exogenous/endogenous luminescence substance (for example, any luminescence agent, any natural luminescence component, based on any luminescence phenomenon, such as fluorescence, phosphorescence, chemiluminescence, bio-luminescence, induced Raman-radiation, and so on); moreover, the further reflectance images may be of any type (for example, either the same or different with respect to the reflectance images).

In an embodiment, the method comprises determining (by the computing device) the correction for each registration pair of reflectance image and luminescence image according to the corresponding reflectance images and further reflectance images. However, the possibility of determining the correction according to the reflectance images and the luminescence images is not excluded.

In an embodiment, the method comprises registering (by the computing device) each registration pair of reflectance image and luminescence image according to the corresponding correction. However, the possibility of registering each registration pair of reflectance image and further reflectance image as well is not excluded.

In an embodiment, the method comprises outputting (on the output unit) the representation of the body-part based on the reflectance image and the luminescence image of each registration pair being registered. However, the possibility of outputting the representation of the body-part based on the (registered) further reflectance images as well is not excluded.

In an embodiment, the method comprises acquiring (with the second endoscopic unit) the second sequence of second image sets being corresponding luminescence images representative of a luminescence light emitted in the second field of view by a luminescence substance. However, the luminescence images may be of any type (see above).

In an embodiment, the method comprises illuminating (by the second endoscopic unit) the second field of view with an excitation light for the luminescence substance being a fluorescence substance. However, the excitation light may be of any type (for example, NIR, visible and so on) for any fluorescence substance (for example, extrinsic or intrinsic, exogenous or endogenous, and so on).

In an embodiment, the method comprises acquiring (with the second endoscopic unit) the second sequence of second image sets comprising the corresponding luminescence images being corresponding fluorescence images representative of the luminescence light being a fluorescence light emitted in the second field of view by the fluorescence substance in response to the excitation light. However, the fluorescence light may be of any type (for example, NIR, Infra-Red (IR), visible and so on).

In an embodiment, the luminescence substance is a luminescence agent being pre-administered to the patient before performing the method. However, the luminescence agent may be of any type (for example, any targeted luminescence agent, such as based on specific or non-specific interactions, any non-targeted luminescence agent and so on) and it may have been pre-administered in any way (for example, with a syringe, an infusion pump, and so on) and at any time (for example, few or some hours/days in advance, immediately before performing the method, continuously during it and so on); moreover, the luminescence agent may also be administered to the patient in a non-invasive manner (for example, orally for imaging the gastrointestinal tract, via a nebulizer into the airways, via topical spray application or topical introduction during a surgical procedure, and so on), and in any case without any substantial physical intervention on the patient that would require professional medical expertise or entail any health risk for him/her (for example, intramuscularly).

In an embodiment, the correction is entered manually according to a display of the first images and the second images. However, the correction may be entered according to any display of the first/second images (for example, with the first/second images that are played during their acquisition or are paused, at the beginning and possibly at any next time, and so on).

In an embodiment, the method comprises acquiring (with the first endoscopic unit) the first sequence of first images being in color. However, the first images may be represented in color in any way (for example, in any color space, with any color model and so on).

In an embodiment, the method comprises acquiring (with the second endoscopic unit) the second sequence of second image sets being in color. However, the second images may be represented in color in any way (for example, either the same or different with respect to the first images).

In an embodiment, the method comprises preparing (by the computing device) the first images and the second images for said registering by conversion to gray scale. However, the images may be converted to grayscale in any way (for example, based on average methods, weighted methods and so on).

In an embodiment, the method comprises preparing (by the computing device) the first images and the second images for said registering by down-sampling. However, the images may be down-sampled by any rate and in any way (for example, simply by averaging their values, applying smoothing filters and so on).

In an embodiment, the method comprises preparing (by the computing device) the first images and the second images for said registering by limiting to corresponding central portions. However, the images may be limited to any central portions (for example, with any size, shape and so on).

An embodiment provides a method for training the neural network of above. However, the (training) method may be applied to the neural network for any purpose (for example, for its development, maintenance, verification and so on).

In an embodiment, the method comprises the following steps under the control of a computing system. However, the computing system may be of any type (see below).

In an embodiment, the method comprises providing (to the computing system) a plurality of first sample images of at least one sample body-part of at least one sample patient. However, the first sample images may be in any number, relating to any number and type of sample body-parts of any number and type of sample patients and acquired with any number and type of sample endoscopic units inserted into any type of sample cavity of the sample body-part (for example, either the same or different with respect to above).

In an embodiment, the method comprises synthesizing (by the computing system) corresponding synthetic images from the first sample images. However, the synthetic images may be synthesized in any way (for example, by changing shape, changing resolution, changing contrast, zooming in/out, translating, adding/removing noise, any combination thereof and so on).

In an embodiment, the method comprises synthesizing (by the computing system) the synthetic images by changing a shape of the corresponding first sample images. However, the shape may be changed in any way (for example, from rectangular, polygonal, circular and so on to circular, rectangular, polygonal and so on).

In an embodiment, the method comprises synthesizing (by the computing system) the synthetic images by reducing a resolution of the corresponding first sample images. However, the resolution may be reduced by any extent and in any way (for example, by down-sampling, filtering and so on).

In an embodiment, the method comprises synthesizing (by the computing system) the synthetic images by reducing a contrast of the corresponding first sample images. However, the contrast may be reduced by any extent and in any way (for example, by saturating, applying histogram equalization with a worsening distribution, either at the level of the whole images or of small tiles thereof, and so on).

In an embodiment, the method comprises synthesizing (by the computing system) the synthetic images by zooming in the corresponding first sample images. However, the first sample images may be zoomed in by any extent and in any way (for example, by replicating values, applying smoothing filters and so on).

In an embodiment, the method comprises synthesizing (by the computing system) the synthetic images by translating the corresponding first sample images. However, the first sample images may be translated by any extent (for example, by pre-defined values, in a random way and so on).

In an embodiment, the method comprises synthesizing (by the computing system) the synthetic images by adding noise to the corresponding first sample images. However, the noise may be added in any way (for example, by adding a pre-defined or random noise to the first sample images uniformly or in a random way, and so on).

In an embodiment, the method comprises generating (by the computing system) a plurality of second sample images each by applying a corresponding reference motion to one of the synthetic images. However, the second sample images may be generated in any number and in any way (for example, by using any number and type of reference motions, such as translation, rotation, their combination and the like, by applying all the possible reference motions or corresponding subsets thereof selected randomly to each synthetic image, and so on).

In an embodiment, the method comprises training (by the computing system) the neural network according to a plurality of training sets each comprising one of the second sample images, the corresponding first sample image and the corresponding reference motion. However, the training sets may be selected in any number and in any way (for example, randomly, uniformly and so on), and they may be used to train the neural network in any way (for example, based on the Stochastic Gradient Descent, the Real-Time Recurrent Learning, higher-order gradient descent, the Extended Kalman-filtering and so on algorithms), more generally, the training sets may be provided in any other way (for example, by acquiring both the first and second sample images with corresponding sample endoscopic units and then re-aligning each pair of them manually, automatically or semi-automatically with another method, such as based on the above-described optical flow technique, with the possibility of increasing their number with further pairs of sample images generated by applying random motions, by synthesizing both the first sample images and the second sample images and then applying random motions, and so on).

Generally, similar considerations apply if the same solution is implemented with equivalent imaging/training methods (by using similar steps with the same functions of more steps or portions thereof, removing some steps being non-essential, or adding further optional steps); moreover, the steps may be performed in a different order, concurrently or in an interleaved way (at least in part).

An embodiment provides a computer program, which is configured for causing a computing device to perform a method for operating an endoscopic system to image a body-part of a patient when the computer program is executed on the computing device. However, the (operating) method may be used for imaging any body-part and of any patient in any medical procedure (see above); moreover, the computer program may run on any computing device (see below).

In an embodiment, the method comprises receiving a first sequence of a plurality of first images of a first field of view containing at least part of the body-part. However, the first images may be in any number and of any type (see above); moreover, the first images may be received in any way (for example, transferred via a wired/wireless interface, acquired directly, transferred with a removable storage unit, downloaded from a network and so on).

In an embodiment, the method comprises receiving a second sequence of a plurality of second image sets corresponding to the first images, each of the second image sets comprising one or more second images of a second field of view containing at least part of the body-part. However, the first images and the second image sets may correspond in any way, each second image set may comprise any number of second images of any type, and the first field of view and the second field of view may be of any type (see above); moreover, the second images may be received in any way (for example, acquired directly, transferred via a wired/wireless interface or with a removable storage unit, downloaded from a network and so on).

In an embodiment, the method comprises estimating a first motion of a first probe, of a first endoscopic unit being used to acquire the first sequence of first images, and a second motion of a second probe, of a second endoscopic unit being used to acquire the second sequence of second image sets, independently for each current one of the first images and for a current second image of each current one of the second image sets, respectively, the first motion being estimated according to an estimation set of a plurality of the first images corresponding to the current first image and the second motion being estimated according to an estimation set of a plurality of the second images corresponding to the current second image. However, the motions may be of any type, indicated in any way, estimated in any way according to any type of estimation set of first/second images (see above).

In an embodiment, the method comprises determining at least one correction for each registration pair of a first image of the first images and a second image of the corresponding second image set according to the first motions and the second motions of a calculation set of one or more of the first images and of the second images, respectively, corresponding to the pair of first image and second image. However, the correction may be of any type and it may be determined in any way (see above).

In an embodiment, the method comprises registering each registration pair of first image and second image according to the corresponding correction. However, the registration may be of any type, performed in any way and at any time (see above).

In an embodiment, the method comprises outputting a representation of the body-part based on the first image and the second image of each registration pair being registered. However, the representation of the body-part may be output in any way, on any output unit and based on the registered images in any way (see above).

An embodiment provides a computer program product comprising a computer readable storage medium embodying a computer program, the computer program being loadable into a working memory of a computing device thereby configuring the computing device to perform the same (operating) method when the computer program is executed on the computing device.

An embodiment provides a computer program configured for causing a computing system to perform the (training) method of above when the computer program is executed on the computing system. However, the computer program may run on any computing system (see below).

An embodiment provides a computer program product comprising a computer readable storage medium embodying a computer program, the computer program being loadable into a working memory of a computing system thereby configuring the computing system to perform the same (training) method when the computer program is executed on the computing system.

Generally, each (computer) program may be implemented as a stand-alone module, as a plug-in for a pre-existing software program (for example, an imaging application of the computing device for the operating method or a configuration application of the computing system for the training method) or even directly in the latter. In any case, similar considerations apply if the program is structured in a different way, or if additional modules or functions are provided; likewise, the memory structures may be of other types or may be replaced with equivalent entities (not necessarily consisting of physical storage media). The program may take any form suitable to be used by any computing device/system (see below), thereby configuring the computing device/system to perform the desired operations; particularly, the program may be in the form of external or resident software, firmware, or microcode (either in object code or in source code, for example, to be compiled or interpreted). Moreover, it is possible to provide the program on any computer readable storage medium. The storage medium is any tangible medium (different from transitory signals per se) that may retain and store instructions for use by the computing device/system. For example, the storage medium may be of the electronic, magnetic, optical, electromagnetic, infrared, or semiconductor type; examples of such storage medium are fixed disks (where the program may be pre-loaded), removable disks, memory keys (for example, of USB type) and the like. The program may be downloaded to the computing device/system from the storage medium or via a network (for example, the Internet, a wide area network and/or a local area network comprising transmission cables, optical fibers, wireless connections, network devices); one or more network adapters in the computing device/system receive the program from the network and forward it for storage into one or more storage devices of the computing device/system. In any case, the solution according to an embodiment of the present disclosure lends itself to be implemented even with a hardware structure (for example, by electronic circuits integrated in one or more chips of semiconductor material, such as of Field Programmable Gate Array (FPGA) or Application-Specific Integrated Circuit (ASIC) type), or with a combination of software and hardware suitably programmed or otherwise configured.

An embodiment provides an endoscopic system for performing the steps of the (imaging) method of above. However, the endoscopic system may be of any type (for example, with endoscopic units connected together and one of them comprising the computing device and the output unit, with the endoscopic units connected to a separate computing device and output unit, with all the components integrated together and so on).

In an embodiment, the endoscopic system comprises the first endoscopic unit for acquiring the first sequence of first images. However, the first endoscopic unit may be of any type (for example, a motherscope, an independent endoscope, based on any number and type of lenses, wave guides, mirrors, sensors, and so on).

In an embodiment, the endoscopic system comprises the second endoscopic unit for acquiring the second sequence of second image sets. However, the second endoscopic unit may be of any type (for example, a babyscope, an independent endoscope, based on any number and type of lenses, wave guides, mirrors, sensors, and so on).

In an embodiment, the endoscopic system comprises the computing device for registering each registration pair of first image and second image. However, the computing device may be of any type (see below).

In an embodiment, the endoscopic system comprises the output unit for outputting the representation of the body-part. However, the output unit may be of any type (for example, a monitor, virtual reality glasses, a printer and so on).

An embodiment provides an endoscopic equipment (for use in the endoscopic system of above) which comprises one between the first endoscopic unit and the second endoscopic unit. However, the endoscopic equipment may be of any type (for example, comprising the babyscope, the motherscope and so on).

In an embodiment, said endoscopic unit comprises an acquisition unit for acquiring a corresponding one between the first sequence of first images and the second sequence of second image sets. However, the acquisition unit may be of any type (for example, based on CCD, ICCD, EMCCD, CMOS, InGaAs or PMT sensors, with any illumination unit for applying the excitation light, such as based on laser, LEDs, UV lamps and the like, and/or the white light, such as with LEDs, halogen/Xenon lamps and the like, to the body-part, and so on).

In an embodiment, said endoscopic unit comprises an interface for receiving the other one between the first sequence of first images and the second sequence of second image sets from the other one of the first endoscopic unit and the second endoscopic unit. However, the interface may be of any type (for example, wired, wireless, serial, parallel and so on).

In an embodiment, said endoscopic unit comprises the computing device for registering each registration pair of first image and second image. However, the computing device may be of any type (see below).

In an embodiment, said endoscopic unit comprises the output unit for outputting the representation of the body-part. However, the output unit may be of any type (see above).

An embodiment provides a computing device, which comprises means configured for performing the steps of the (operating) method of above. An embodiment provides a computing device comprising a circuit (i.e., any hardware suitably configured, for example, by software) for performing each step of the same (operating) method. However, the computing device may be of any type (for example, a central unit of each endoscopic unit that receives the other images from the other endoscopic unit, a common central unit of the endoscopic system for both the endoscopic units, a separate computer that receives the corresponding images from the two endoscopic units or all the images from the endoscopic system, and so on).

An embodiment provides a computing system, which comprises means configured for performing the steps of the (training) method of above. An embodiment provides a computing system comprising a circuit (i.e., any hardware suitably configured, for example, by software) for performing each step of the same (training) method. However, the computing system may be of any type (for example, a personal computer, a server, a virtual machine, such as provided in a cloud environment, and so on).

Generally, similar considerations apply if the endoscopic system, the endoscopic equipment, the computing device and the computing system each has a different structure or comprises equivalent components, or it has other operative characteristics. In any case, every component thereof may be separated into more elements, or two or more components may be combined together into a single element; moreover, each component may be replicated to support the execution of the corresponding operations in parallel. Moreover, unless specified otherwise, any interaction between different components generally does not need to be continuous, and it may be either direct or indirect through one or more intermediaries.

An embodiment provides a surgical method comprising the following steps. A body-part of a patient is imaged by performing the (imaging) method of above thereby outputting the representation of the body-part during a surgical procedure of the body-part. The body-part is operated according to the outputting of the representation thereof. However, the proposed method may find application in any kind of surgical method in the broadest meaning of the term (for example, for curative purposes, for prevention purposes, for aesthetic purposes, and so on) and for acting on any kind of body-part of any patient (see above).

An embodiment provides a diagnostic method comprising the following steps. A body-part of a patient is imaged by performing the (imaging) method of above thereby outputting the representation of the body-part during a diagnostic procedure of the body-part. A health condition of the body-part is evaluated according to the outputting of the representation thereof. However, the proposed method may find application in any kind of diagnostic applications in the broadest meaning of the term (for example, aimed at discovering new lesions, at monitoring known lesions, and so on) and for analyzing any kind of body-part of any patient (see above).

An embodiment provides a therapeutic method comprising the following steps. A body-part of a patient is imaged by performing the (imaging) method of above thereby outputting the representation of the body-part during a therapeutic procedure of the body-part. The body-part is treated according to the outputting of the representation thereof. However, the proposed method may find application in any kind of therapeutic method in the broadest meaning of the term (for example, aimed at curing a pathological condition, at avoiding its progress, at preventing the occurrence of a pathological condition, or simply at ameliorating a comfort of the patient) and for acting on any kind of body-part of any patient (see above).

What is claimed is:

1. A method for imaging a body-part of a patient with an endoscopic system, wherein the method comprises:
acquiring, with a first endoscopic unit of the endoscopic system, a first sequence of a plurality of first images of a first field of view containing at least part of the body-part via a first probe of the first endoscopic unit,
acquiring, with a second endoscopic unit of the endoscopic system, a second sequence of a plurality of second image sets corresponding to the first images, each of the second image sets comprising one or more second images of a second field of view containing at least part of the body-part, via a second probe of the second endoscopic unit being movable with respect to the first probe,
registering, by a computing device, each registration pair of a first image of the first images and a second image of the corresponding second image set, and
outputting, on an output unit, a representation of the body-part based on the first image and the second image of each registration pair being registered.

2. The method according to claim 1, wherein the method comprises:
acquiring, with the second endoscopic unit, the second sequence of second image sets via the second probe-inserted removably into a working channel of the first endoscopic unit.

3. The method according to claim 1, wherein the computing device is comprised in one between the first endoscopic unit and the second endoscopic unit, the method comprising:
receiving, by the computing device, the corresponding first sequence of first images or second sequence of second image sets from the other one of the first endoscopic unit and the second endoscopic unit.

4. (canceled)

5. The method according to claim 41, wherein the method comprises:
estimating, by the computing device, a first motion of the first probe and a second motion of the second probe independently for each current one of the first images and for a current second image of each current one of the second image sets, respectively, the first motion being estimated according to an estimation set of a plurality of the first images corresponding to the current first image and the second motion being estimated according to an estimation set of a plurality of the second images corresponding to the current second image, and
determining, by the computing device, a correction due to a corresponding relative movement of the first probe and the second probe for each registration pair of first image and second image according to the first motions and the second motions of a calculation set of one or more of the first images and of the second images, respectively, corresponding to the pair of first image and second image, and
registering, by the computing device, each registration pair of first image and second image according to the corresponding correction.

6. (canceled)

7. The method according to claim 5, wherein the method comprises:
calculating, by the computing device, a misalignment for each pair of corresponding current first image and current second image according to the corresponding first motion and second motion, and
calculating, by the computing device, the correction for each registration pair of first image and second image according to the misalignments of the corresponding calculation set of first images and second images being weighted decreasingly with corresponding extents and/or with corresponding temporal distances from the registration pair of first image and second image.

8. The method according to claim 1, wherein the method comprises:
supplying, by the computing device, a neural network with input data based on an estimation set of first images and an estimation set of second images for each registration pair of first image and second image,
receiving, by the computing device, a correction for each registration pair of first image and second image from the neural network due to a corresponding relative movement of the first probe and the second probe, and
registering, by the computing device, each registration pair of first image and second image according to the corresponding correction.

9. The method according to claim 86, wherein the method comprises:
selecting, by the neural network, the correction for each registration pair of first image and second image among a plurality of pre-defined corrections.

10. The method according to claim 7, wherein the neural network is a convolutional neural network, the convolutional neural network comprising, along a processing direction of the convolutional neural network, a plurality of groups, each comprising one or more convolutional layers followed by a max-pooling layer, and a plurality of fully connected layers providing corresponding probabilities of the pre-defined corrections.

11. (canceled)

12. The method according to claim 1, wherein the method comprises:
receiving, by the computing device, a correction due to a corresponding relative movement of the first probe and the second probe being entered manually according to a display of the first images and the second images, and
registering, by the computing device, each registration pair of first image and second image according to the corresponding correction.

13. The method according to claim 1, wherein the method-comprises:
acquiring, with the first endoscopic unit, the first sequence of first images being corresponding reflectance images representative of a visible light reflected by a content of the first field of view,
acquiring, with the second endoscopic unit, the second sequence of second image sets being corresponding luminescence images representative of a luminescence light emitted in the second field of view by a luminescence substance and corresponding further reflectance images representative of the visible light reflected by a content of the second field of view,
determining, by the computing device, a correction due to a corresponding relative movement of the first probe and the second probe for each registration pair of reflectance image and luminescence image according to the corresponding reflectance images and further reflectance images,
registering, by the computing device, each registration pair of reflectance image and luminescence image according to the corresponding correction, and
outputting, on the output unit, the representation of the body-part based on the reflectance image and the luminescence image of each registration pair being registered.

14. The method according to claim 1, wherein the method comprises:
acquiring, with the first endoscopic unit, the first sequence of first images being corresponding reflectance images representative of a visible light reflected by a content of the first field of view, and
acquiring, with the second endoscopic unit, the second sequence of second image sets being corresponding luminescence images representative of a luminescence light emitted in the second field of view by a luminescence substance.

15.-16. (canceled)

17. The method according to claim 1, wherein the method comprises:
acquiring, with the first endoscopic unit, the first sequence of first images being in color,
acquiring, with the second endoscopic unit, the second sequence of second image sets being in color, and
preparing, by the computing device, the first images and the second images for said registering by conversion to gray scale, down-sampling and/or limiting to corresponding central portions.

18. A method (800) for training a neural network for use in a method for imaging a body-part of a patient with an endoscopic system, wherein the method comprises, under the control of a computing system:
providing, to the computing system, a plurality of first sample images of at least one sample body-part of at least one sample patient,
synthesizing, by the computing system, corresponding synthetic images from the first sample images by changing a shape of, reducing a resolution of, reducing a contrast of, zooming in, translating and/or adding noise to the corresponding first sample images,
generating, by the computing system, a plurality of second sample images each by applying a corresponding reference motion to one of the synthetic images, and
training, by the computing system, the neural network according to a plurality of training sets each comprising one of the second sample images, the corresponding first sample image and the corresponding reference motion.

19. (canceled)

20. A computer program product comprising a computer readable storage medium embodying a computer program, the computer program being loadable into a working memory of a computing device thereby configuring the computing device to perform a method for operating an endoscopic system to image a body-part of a patient when the computer program is executed on the computing device, wherein the method comprises:
receiving a first sequence of a plurality of first images of a first field of view containing at least part of the body-part,
receiving a second sequence of a plurality of second image sets corresponding to the first images, each of the second image sets comprising one or more second images of a second field of view containing at least part of the body-part,
estimating a first motion of a first probe, of a first endoscopic unit being used to acquire the first sequence of first images, and a second motion of a second probe, of a second endoscopic unit being used to acquire the second sequence of second image sets, independently for each current one of the first images and for a current second image of each current one of the second image sets, respectively, the first motion being estimated according to an estimation set of a plurality of the first images corresponding to the current first image and the second motion being estimated according to an estimation set of a plurality of the second images corresponding to the current second image,
determining at least one correction for each registration pair of a first image of the first images and a second image of the corresponding second image set according to the first motions and the second motions of a calculation set of one or more of the first images and of the second images, respectively, corresponding to the pair of first image and second image,
registering each registration pair of first image and second image according to the corresponding correction, and
outputting a representation of the body-part based on the first image and the second image of each registration pair being registered.

21. (canceled)

22. A computer program product comprising a computer readable storage medium embodying a computer program, the computer program being loadable into a working memory of a computing system thereby configuring the computing system to perform a method for training a neural network for use in a method for imaging a body-part of a patient with an endoscopic system when the computer program is executed on the computing system, wherein the method comprises:
providing a plurality of first sample images of at least one sample body-part of at least one sample patient,
synthesizing corresponding synthetic images from the first sample images by changing a shape of, reducing a resolution of, reducing a contrast of, zooming in, translating and/or adding noise to the corresponding first sample images,
generating a plurality of second sample images each by applying a corresponding reference motion to one of the synthetic images, and
training the neural network according to a plurality of training sets each comprising one of the second sample images, the corresponding first sample image and the corresponding reference motion.

23. An endoscopic system for imaging a body-part of a patient, wherein the endoscopic system comprises:
a first endoscopic unit having a first probe for acquiring a first sequence of a plurality of first images of a first field of view containing at least part of the body-part,
a second endoscopic unit having a second probe being movable with respect to the first probe for acquiring a second sequence of a plurality of second image sets corresponding to the first images, each of the second image sets comprising one or more second images of a second field of view containing at least part of the body-part,
a computing device for registering each registration pair of a first image of the first images and a second image of the corresponding second image set, and
an output unit for outputting a representation of the body-part based on the first image and the second image of each registration pair being registered.

24. The endoscopic system according to claim 16, wherein one between the first endoscopic unit and the second endoscopic comprise:
an acquisition unit for acquiring a corresponding one between the first sequence of first images and the second sequence of second image sets,
an interface for receiving the other one between the first sequence of first images and the second sequence of second image sets from the other one of the first endoscopic unit and the second endoscopic unit,
the computing device for registering each registration pair of first image and second image, and
the output unit for outputting the representation of the body-part based on the first image and the second image of each registration pair being registered.

25. (canceled)

26. A computing device for operating an endoscopic system to image a body-part of a patient, wherein the computing device comprises:
a circuitry for receiving a first sequence of a plurality of first images of a first field of view containing at least part of the body-part,
a circuitry for receiving a second sequence of a plurality of second image sets corresponding to the first images, each of the second image sets comprising one or more second images of a second field of view containing at least part of the body-part,
a circuitry for estimating a first motion of a first probe, of a first endoscopic unit being used to acquire the first sequence of first images, and a second motion of a second probe, of a second endoscopic unit being used to acquire the second sequence of second image sets, independently for each current one of the first images and for a current second image of each current one of the second image sets, respectively, the first motion being estimated according to an estimation set of a plurality of the first images corresponding to the current first image and the second motion being estimated according to an estimation set of a plurality of the second images corresponding to the current second image,
a circuitry for determining at least one correction for each registration pair of a first image of the first images and a second image of the corresponding second image set according to the first motions and the second motions of a calculation set of one or more of the first images and of the second images, respectively, corresponding to the pair of first image and second image,
a circuitry for registering each registration pair of first image and second image according to the corresponding correction, and
a circuitry for outputting a representation of the body-part based on the first image and the second image of each registration pair being registered.

27. (canceled)

28. A computing system for training a neural network for use in a method for imaging a body-part of a patient with an endoscopic system, wherein the computing system comprises:
a circuitry for providing a plurality of first sample images of at least one sample body-part of at least one sample patient,
a circuitry for synthesizing corresponding synthetic images from the first sample images by changing a shape of, reducing a resolution of, reducing a contrast of, zooming in, translating and/or adding noise to the corresponding first sample images,
a circuitry for generating a plurality of second sample images each by applying a corresponding reference motion to one of the synthetic images, and
a circuitry for training the neural network according to a plurality of training sets each comprising one of the second sample images, the corresponding first sample image and the corresponding reference motion.

29. A medical method comprising:
imaging a body-part of a patient with an endoscopic system by:
acquiring, with a first endoscopic unit of the endoscopic system, a first sequence of a plurality of first images of a first field of view containing at least part of the body-part via a first probe of the first endoscopic unit,
acquiring, with a second endoscopic unit of the endoscopic system, a second sequence of a plurality of second image sets corresponding to the first images, each of the second image sets comprising one or more second images of a second field of view containing at least part of the body-part, via a second probe of the second endoscopic unit being movable with respect to the first probe,
registering, by a computing device of the endoscopic system, each registration pair of a first image of the first images and a second image of the corresponding second image set, and
outputting, on an output unit of the endoscopic system, a representation of the body-part based on the first image and the second image of each registration pair being registered; and
performing a medical procedure relating to the body-part according to the outputting of the representation thereof.

30.-31. (canceled)
